(19) United States
(12) Patent Application Publication
(10) Pub. No.: US 2005/0162335 A1
(43) Pub. Date: Jul. 28, 2005

(54) Plasma device

(75) Inventor: Nobuo Ishii, Amagasaki-Shi, Hyogo (JP)

Correspondence Address: CROWELL & MORING LLP;INTELLECTUAL PROPERTY GROUP, P.O. BOX 14300, WASHINGTON, DC, 20044-4300, US

(73) Assignee: TOKYO ELECTRON LIMITED, Tokyo (JP)

(21) Appl. No.: 10/504,932

(22) PCT Filed: Mar. 8, 2002
(86) PCT No.: PCT/JP02/02212

(51) Int. Cl. A61N 1/00

(52) U.S. Cl. 343/908; 607/129

(57) ABSTRACT

A plasma apparatus includes a patch antenna emitting a high frequency wave into a processing container. The patch antenna has a conductive plate forming a resonator and a ground plane. The conductive plate of the patch antenna is fed such that the emitted high frequency wave becomes a circular polarization.

### TECHNICAL FIELD

The present invention relates to a plasma apparatus generating plasma by a high frequency wave or an electromagnetic field to perform prescribed processes.

### BACKGROUND ART

Plasma apparatuses are widely used in manufacturing semiconductor devices, to perform processes such as oxide film formation, crystal growth of a semiconductor layer, etching and ashing. Among such plasma apparatuses, there is a high frequency plasma apparatus that supplies a high frequency wave into a processing container using an antenna, and generates high-density plasma. The high frequency plasma apparatus is capable of generating plasma stably even when plasma gas pressure is relatively low, and hence, application thereof is wide.

Recently, the use of a patch antenna in this high frequency plasma apparatus has been studied as an antenna for supplying a high frequency wave into the processing container. FIGS. 37A-37C shows an exemplary configuration of a conventional patch antenna used in the high frequency plasma apparatus. FIG. 37A is a plan view of the patch antenna viewed from the emitting surface, FIG. 37B is a cross section along a line XXXVIIB-XXXVIIB of FIG. 37A, and FIG. 37C shows coordinate systems corresponding to FIG. 37A.

As shown in FIG. 37B, the patch antenna has a ground plane 531 formed of a grounded conductive plate, and a conductive plate 532 forming a resonator. Ground plane 531 and conductive plate 532 are provided at opposing faces of a dielectric plate 534, respectively. Conductive plate 532 is connected at its center O to ground plane 531 by a conductive line 533 that penetrates dielectric plate 534.

As shown in FIG. 37A, the two-dimensional shape of conductive plate 532 is a rectangle, in which the length of the longer edge is L1 and that of the shorter edge is L2 (L2<L1). When the wavelength of the electromagnetic field in the patch antenna is given as λg, the length of the longer edge L1 is set to L1≈λg/2. For ease of description, x axis and y axis are assumed to be parallel to the longer edge and the shorter edge of conductive plate 532, respectively, and the origin of the coordinate systems is assumed to be at center O of conductive plate 532.

As shown in FIG. 37B, the patch antenna is connected to a high frequency power source 545 through a coaxial line 541. Here, an outer conductor 542 of coaxial line 541 is connected to ground plane 531, while an inner conductor 543 of coaxial line 541 penetrates an opening of ground plane 542 and dielectric plate 534 to be connected to conductive plate 532 at a point PP on x axis.

FIGS. 38A and 38B are for describing the principle of radiation of the electromagnetic field by the patch antenna. Here, FIG. 38 A shows conductive plate 532, while FIG. 38B shows a current distribution (dotted line) and a voltage distribution (solid line) in x axis direction in conductive plate 532.

As the length of the longer edge L1 is approximately λg/2, the current supplied from high frequency power supply 545 to conductive plate 532 resonates in the longer edge direction, i.e., in the x axis direction, to be a standing wave, of which current distribution takes the form of sine wave where the opposing ends are fixed to 0 (zero), as indicated by the dotted line in FIG. 38B. When the current resonates and becomes such a standing wave, the waveform of the voltage and the waveform of the current have different phases from each other by 90°.

In the state shown in FIG. 38B, the voltage at the left end of conductive plate 532 is positive, and therefore the line of electric force is directed from conductive plate 532 to ground plane 531. On the other hand, the voltage at the right end of conductive plate 532 is negative, and therefore the line of electric force is directed from ground plane 531 to conductive plate 532. As the direction of the line of electric force is the same as a displacement current, magnetic currents flow in the same direction along the left and right ends of conductive plate 532, as shown in FIG. 38A. As an electromagnetic field is emitted having the magnetic currents as the wave source, the electromagnetic field forms a TM10 mode in which the magnetic field is parallel to y axis.

FIGS. 39A and 39B are conceptual illustrations of field intensity distributions formed by the patch antenna. FIG. 39A indicates the field intensity distribution in the xz plane, while FIG. 39B indicates the field intensity distribution in the yz plane.

As above, as the electromagnetic field emitted by the patch antenna forms the TM10 mode in which the magnetic field is parallel to y axis, the field intensity distributions thereof show characteristics similar to a dipole antenna, as shown in FIGS. 39A and 39B. Specifically, while it is relatively uniform in the xz plane as in FIG. 39A, large bias occurs in yz plane as in FIG. 39B. The field intensity distribution in the yz plane shows the greatest electric field at center O of conductive plate 532, and electric field is weakened noticeably as distanced therefrom.

Accordingly, when plasma is generated in an electromagnetic field with such a spatial distribution, the field intensity being higher on x axis than the surroundings results in the plasma density directly under x axis being higher than the surroundings. Accordingly, there has been a problem that when an etching apparatus is formed with a conventional plasma processing apparatus using a single patch antenna of which operation is similar to a dipole, the processing speed thereof varies depending on the location, i.e., the etching speed becomes faster in the area immediately under x axis where the plasma density is high. This is the first problem.

Recently, the processing container tends to have a larger diameter as the size of a wafer or an LCD (liquid crystal display) increases. As the diameter of the processing container increases from the size corresponding to a half wavelength (λg/2) of the high frequency wave used to the size corresponding to one wavelength (λg), and further increases more than the size corresponding to one wavelength, a standing wave is generated in the processing container in the radial direction or the peripheral direction. The electric field is great at the antinode of the standing wave, while it is small at the node thereof, therefore the standing wave generated in the processing container hinders control of plasma to be uniform. Accordingly, when making the diameter of the processing container larger, it is necessary to set the wavelength of high frequency wave used longer correspondingly so that the standing wave would not be generated.

For avoiding the generation of the standing wave in the processing container, there is a conventional example to use a dipole antenna as an antenna for supplying the electromagnetic field of high frequency into the processing container. FIG. 40 shows a plan view of the dipole antenna.

The dipole antenna 3530 is arranged on a dielectric plate 3512 that isolates the antenna from a processing container (not shown) generating plasma, and formed with two conductive poles 3531, 3532 arranged linearly and parallel to the main surface of dielectric plate 3512. The opposing ends of conductive poles 3531, 3532 are away from each other, and connected to a high frequency power source 545 for feeding electricity. In order to emit an intense high frequency wave using a resonance phenomenon, dipole antenna 3530 needs to have a length of an odd multiple of a half wavelength (i.e., (2N−1)×λg/2, where λg is the wavelength of the electromagnetic field above dipole antenna 3530 and N is a natural number).

Dipole antenna 3530 can only be used with the processing container having a diameter L approximately larger than λg/2 due to its antenna size of (2N−1)×λg/2. Conversely, if dipole antenna 3530 is employed, with the processing container with diameter L, the high frequency wave having a wavelength approximately higher than 2 L can not be used (in other words, the high frequency wave having a wavelength approximately lower than c/(2 L) (where c is the speed of light) can not be used). As above, there has been a problem that the use of dipole antenna 3530 in the plasma apparatus limits the diameter of a suitable processing container, or the frequency of the high frequency wave. This is the second problem.

FIG. 41 shows an exemplary configuration of an etching apparatus using a conventional high frequency plasma apparatus. FIGS. 42A and 42B show the configuration of a patch antenna used in the etching apparatus. Here, FIG. 42A is a plan view of patch antenna 4530 shown in FIG. 41 viewed from the bottom, while FIG. 42B shows coordinate systems.

In the etching apparatus shown in FIG. 41, a sealed container is formed by a cylindrical processing container 511 open at the upper portion, and a dielectric plate 512 closing the upper opening of processing container 511. At the bottom of processing container 511, an exhaust port 515 is provided for vacuum evacuation. On a sidewall of processing container 511, a processing gas supply nozzle 517 is provided for introducing etching gas. In processing container 511, a mounting table 522 is accommodated for placing a substrate 521 to be etched. To mounting table 522, a high frequency power source 526 for bias is connected.

To the upper portion of dielectric plate 512, a patch antenna 4530 is arranged for supplying a high frequency electromagnetic field into processing container 511 through dielectric plate 512. Shield member 518 covers the periphery of dielectric plate 512 and antenna 4530. Antenna 4530 is connected to high frequency power source 545 for feeding electricity.

Patch antenna 4530 has a ground plane 4531 formed with a grounded conductive plate, and a conductive plate 4532 arranged facing to ground plane 4531 to form a resonator (hereinafter referred to as a patch). As shown in FIG. 42A, patch 4532 is in a circular shape (two-dimensionally) having a diameter L1≈λg. λg is a wavelength of the electromagnetic field between patch 4532 and ground plane 4531. Here, it is assumed that patch 4532 is on the xy plane of which center O is the origin of the coordinate systems.

As shown in FIG. 41, in patch antenna 4530, a feed point is provided at center O of patch 4532. Coaxial line 541 is used for feeding antenna 4530, of which outer conductor 542 is connected to ground plane 4531, while inner conductor 543 is connected to center O of patch 4532.

Additionally, patch 4532 is connected to ground plane 4531 through short pins 4533 at three points P1, P2, P3, which are isotropically away from center O by approximately λg/4. It is assumed that point P1 is on x axis.

FIGS. 43A and 43B shows the operating principle of patch antenna 4530. Diameter L1 of patch 4532 is approximately λg, and therefore the current supplied from high frequency power source 545 to center O of patch 4532 oscillates to be a standing wave. As shown in FIG. 43B, the voltage waveform at this time on x axis becomes an antinode at center O, which is the feeding point, and becomes a node at point P1 that is grounded. At the periphery of patch 4532 the voltage changes at the same phase, therefore as shown in FIG. 43A, magnetic currents generated along the periphery of patch 4532 take the same direction along the entire periphery as viewed from center O. Accordingly, with patch antenna 4530, the TM01 mode is excited, but not the TM11 mode.

Patch antenna 4530 emits a high frequency wave with the above described magnetic currents as the wave source. The electromagnetic field of this high-frequency wave supplied into processing container 511 through dielectric plate 512 causes electrolytic dissociation of a gas in processing container 511, to generate plasma in an upper space 550 above substrate 521 to be processed. The plasma diffuses in processing container 511, and the energy, anisotropy or the like thereof is controlled by the bias voltage applied to mounting table 522 to be used in the etching process.

However, when patch antenna 4530 is in the TM01 mode, the directivity of the high frequency electromagnetic field will be, as shown in FIG. 41, horizontal that is parallel to the main surface of patch 4532 (i.e., the xy plane). Accordingly, a large amount of electricity will be converted to thermal energy at shield member 518 or at processing container 511 before contributing to the generation of plasma. Therefore, there has been a problem that plasma can not be generated effectively. This is the third problem.

### DISCLOSURE OF THE INVENTION

First and second inventions are made to solve the first problem. Specifically, the object thereof is to enable a uniform plasma processing as compared to the conventional manner.

A third invention is made to solve the second problem. Specifically, the object thereof is to increase the degree of freedom in designing a plasma apparatus that has been limited by the size of the antenna.

A fourth invention is made to solve the third problem. Specifically, the object thereof is to increase the efficiency of power at plasma generation.

To provide a plasma apparatus that can perform plasma processing of higher quality by solving these first to third problems is the common object of the first to fourth inventions.

In order to achieve the objects above, a plasma apparatus according to the first invention is characterized by an antenna emitting a high frequency wave into a processing container having a conductive plate arranged facing to a mounting table arranged in the processing container to form a resonator, and a ground plane arranged facing to the conductive plate at a side opposite to the mounting table. The conductive plate is fed such that the emitted high frequency wave becomes a circular polarization. By making the high frequency wave emitted from the antenna to be a circular polarization, the spatial distribution of an electromagnetic field inside the processing container becomes uniform as compared to the conventional manner. The high frequency emitted from the antenna may not be a perfectly circular polarization, and it may be a circular polarization with a polarization ratio of at least 50%, preferably 70%.

Here, two feeding lines may be used for feeding the conductive plate. Specifically, the circular polarization may be generated by two-point feeding.

In this case, the two feeding lines may feed such that that two linear polarizations equal in amplitude, having different phases from each other by 90°, and spatially perpendicular to each other are emitted, respectively. Thus, the high frequency wave emitted from the antenna becomes a circular polarization, which results in even uniform spatial distribution of the electromagnetic field in the processing container.

Further, when the conductive plate has a 90° rotational symmetric form, the two feeding lines may be connected to the conductive plate at two points thereon away from a center of the conductive plate by substantially equal distances and in two directions perpendicular to each other as viewed from the center, for feeding in equal amplitude and with phases different from each other by 90°, respectively.

Still further, in the plasma apparatus described above, one feeding line may be used for feeding the conductive plate of the antenna. Specifically, the circular polarization may be generated by one-point feeding. In this case, the two-dimensional shape of the conductive plate may be a shape having two different lengths in two directions perpendicular to each other as viewed from the center thereof. The feeding line may be connected to the conductive plate at one point thereon in a direction between the two directions. In this case, the conductive plate may have a two-dimensional shape of a circle of which peripheral region is cut out, or an ellipse or a quadrangle.

In order to achieve such an object, a plasma processing apparatus according to the present invention is characterized by an antenna arranged facing to a mounting surface of a mounting table arranged in a processing apparatus for supplying an electromagnetic field of a high frequency wave into the processing container is formed of a plurality of monopole antennas, and configured such that the electromagnetic field forms a circular polarization. Thus, by rotating the electromagnetic field around the axis perpendicular to the mounting surface of mounting table, the plasma distribution generated by the electromagnetic field rotates as well. Thus, the uniformity of plasma distribution when averaged by time can be improved.

Here, the electromagnetic field emitted from the antenna may not be a perfectly circular polarization, and it may be a circular polarization with a polarization ratio of at least 50%, preferably 70%.

Further, a plasma processing apparatus according to the present invention is characterized by an antenna arranged facing to a mounting surface of a mounting table arranged in a processing apparatus for supplying an electromagnetic field of a high frequency wave into the processing container is formed of a plurality of monopole antennas, and configured such that the electromagnetic field forms a substantial TM01 mode. Since the electric field in the substantial TM01 mode is distributed approximately radially around an axis perpendicular to the mounting surface of the mounting table, the uniformity of the plasma distribution in a plane parallel to the mounting surface can be improved.

It is preferable to use a patch antenna for the monopole antenna. By using the patch antenna, the magnetic current forming portion can be made longer to improve the radiation efficiency of the electromagnetic field.

The patch antenna includes a conductive plate arranged facing to the mounting surface of the mounting table, a ground plane arranged facing to the conductive plate at a side opposite to the mounting table as viewed from the conductive plate, a conductive member connecting one end of the conductive plate to the ground plane, and a feeding line connected to the conductive plate at a point away from the one end of the conductive plate. The one end of said conductive plate of said patch antenna, which is connected to the ground plane through the conductive member, may be substantially straight, and a length in a direction perpendicular to the one end may be at most approximately ¼ of a wavelength of the electromagnetic field in the patch antenna.

Alternatively, the patch antenna includes a conductive plate arranged facing to the mounting surface of the mounting table and having substantially straight one end and arc-shaped other end opposing to the one end, a length between the one end and the other end being at most (1.17±0.05)/4 of a wavelength of the electromagnetic field in the patch antenna; a ground plane arranged facing to the conductive plate at a side opposite to the mounting table as viewed from the conductive plate; a conductive member connecting the one end of the conductive plate to the ground plane; and a feeding line connected to the conductive plate at a point away from the one end of the conductive plate. As used herein, substantially straight means a concept including not only a straight line but also a gentle curve.

Further, the conductive plate of the patch antenna may have the other end, which is opposing to the one end connected to the conductive member, folded toward the ground plane, and the conductive member of the patch antenna may have one length in a same direction as the one end of the conductive plate formed shorter than the one end. With such a configuration, the patch antenna may be made smaller.

Still further, the antenna includes at least two conductive plates arranged on a same plane facing to the mounting surface of the mounting table and each having two ends parallel to each other, the length between the two ends being at most approximately ¼ of a wavelength of the electromagnetic field in the antenna; and a ground plane arranged facing to the conductive plate at a side opposite to the mounting table as viewed from the conductive plates; at least two conductive members connecting respective ends of the conductive plates to the ground plane; and at least two feeding lines connected to respective conductive plates at points away from respective ends of the conductive plates. Conductive plates of the antenna are arranged such that the other end of one conductive plate and the other end of the other conductive plate are perpendicular to each other. Feed lines of the antenna feed with phases different from each other. Thus, an electromagnetic field of a circular polarization can be supplied into the processing container.

Still further, the antenna includes a plurality of conductive plates arranged on a same plane facing to the mounting surface of the mounting table and each having substantially straight end, the length in a direction perpendicular to the one end being at most approximately ¼ of a wavelength of the electromagnetic field in the antenna; and a ground plane arranged facing to the conductive plates at a side opposite to the mounting table as viewed from the conductive plates; a plurality of conductive members connecting respective ends of the conductive plates to the ground plane; and a plurality of feeding lines connected to respective conductive plates at points away from respective ends of the conductive plates. The plurality of conductive plates of the antenna are arranged with equal intervals around the center of the antenna with respective one end oriented inward and respective other end, which are opposite to the one end, oriented outward. The plurality of feeding lines of the antenna feed corresponding conductive plate with the same phase. Thus, an electromagnetic field of substantial TM01 mode can be supplied inside the processing container.

In order to achieve the object above, a plasma apparatus according to the present invention is characterized by an forming an antenna for supplying an electromagnetic field of a high frequency wave into a processing container by a monopole antenna. When a wavelength of the electromagnetic field on the antenna is given as λg, then the monopole antenna can be formed with the size of approximately λ/4, at most, and it can still emit a high frequency wave equivalent to that emitted by a dipole antenna. Accordingly, a processing container having a diameter L of less than λg/2 or a high frequency wave of which frequency is lower than approximately c/(2 L) (where c is the speed of light) can be utilized.

Here, it is preferable to use a patch antenna as the monopole antenna. By using the patch antenna, the magnetic current forming portion can be made longer to improve the radiation efficiency of the high frequency wave.

The patch antenna may include a conductive plate arranged facing to a mounting table and having substantially straight one end, a length in a direction perpendicular to the one end being at most approximately ¼ of a wavelength of an electromagnetic field in the patch antenna; a ground plane arranged facing to the conductive plate at a side opposite to the mounting table as viewed from the conductive plate; a conductive material connecting the one end of the conductive plate to the ground plane; and a feeding line connected to a point away from the one end of the conductive plate.

Here, the conductive plate of the patch antenna may have other end opposing to the one end connected to the conductive material folded toward the ground plane. The conductive material of the patch antenna has one length in the same direction as the one end of the conductive plate formed shorter than the one end of the conductive plate. With such a configuration, the patch antenna can be made smaller. Further, a dielectric plate may be arranged between the conductive plate and the ground plate. This shortens the wavelength of the electromagnetic field on the conductive plate to further reduce the size of the antenna.

In order to achieve the object above, a plasma apparatus according to the present invention is characterized by an antenna supplying a high frequency wave into a processing container including a conductive plate arranged facing to the mounting table arranged in the processing container; a ground plane arranged facing to the conductive plate at a side opposite to the mounting table as viewed from the conductive plate; and a plurality of first feeding lines connected to the conductive plate. Two each of the first feeding lines are connected away from each other to at least one first line on the conductive plate perpendicular to a periphery of the conductive plate. Each first line has a length of approximately (N+½)×λg (N is an integer at least 0), where λg is a wavelength of an electromagnetic field between the conductive plate and the ground plane. As used herein, the first line perpendicular to the periphery of the conductive plate is a line parallel to one edge of a quadrangle when the two-dimensional shape of the conductive plate is a quadrangle, and it is a line passing through the center of a circle when the two-dimensional shape of the conductive plate is a circle.

Two first feeding lines are connected on one first line on the conductive plate. As the length of the first line is approximately (N+½)×λg, the currents supplied from these two feeding lines oscillates on the first line to be a standing wave. Here, the feeding performed by the two feeding lines defines the mode of the standing wave. As the voltage waveform on the first line will have antinodes at opposing ends with the number of waves being N+½, the voltage variations at opposing ends show opposite phases with respect to each other. Accordingly, along opposing ends of the first line, magnetic currents are generated in reverse directions with respect to each other as viewed from the center of the conductive plate. Accordingly, in this antenna, the TM11 mode is selectively excited. In TM11 mode, the directivity of a high frequency wave will be perpendicular to the main surface of the conductive plate, and therefore the high frequency wave is oriented directly to mounting table for mounting an object to be processed. Therefore, the power absorbed by the processing container or the like can be reduced to increase the power contributing to the plasma generation.

Here, the first line on the conductive plate forming the antenna may be set to pass through the center of the conductive plate. This prevents the current from passing in the direction perpendicular to the first line on the conductive plate, and therefore the radiation of the high frequency wave in this direction can be suppressed.

Further, in the plasma apparatus above, the antenna may further include a plurality of second feeding lines connected to said conductive plate. Two each of the second feeding lines are connected away from each other to at least one second line on said conductive plate perpendicular to corresponding first line. The length of each second line is approximately (M+½)×λg (M is an integer at least 0). Each second feeding line feeds with a same degree of phase lag behind corresponding first feeding line, such that the high frequency wave becomes a circular polarization. In this case, according to the same principle as above, the TM11 mode is excited also in the second line direction. Further, by making the high frequency wave supplied from the antenna into the processing container to a circular polarization to rotate the electromagnetic field around an axis perpendicular to a mounting surface of a mounting table for mounting an object to be processed, the distribution of plasma generated by the electromagnetic field also rotates. Thus, the uniformity of the plasma distribution when averaged by time can be improved.

Here, the high frequency wave supplied from the antenna is not necessarily a perfectly circular polarization, and it may be a circular polarization with a polarization ratio of at least 50%, preferably at least 70%.

Here, the first and second lines on the conductive plate forming the antenna may be set to pass through the center of the conductive plate. Thus, the current supplied from the first feeding line may not pass through the second line on the conductive plate, and conversely, the current supplied from the second feeding line may not pass through the first line on the conductive plate. Then, generation of a circular polarization reverse to a desired polarization (cross polarization) can be hindered.

Further, the distance between two first feeding lines connected on the same first line, or the distance between two second feeding lines connected on the same second line may be set to λg/2. This would facilitate designing the antenna.

### BRIEF DESCRIPTION OF THE DRAWINGS

FIG. 1 shows a configuration of an etching apparatus according to a first embodiment of the present invention.

FIG. 2A is a plan view showing an exemplary configuration of a conductive plate of a patch antenna shown in FIG. 1.

FIG. 2B shows coordinate systems.

FIGS. 3A and 3B are conceptual illustrations of a time-averaged electric field distribution formed by the patch antenna shown in FIG. 1.

FIG. 4 is a cross sectional view showing a variation of the patch antenna shown in FIG. 1.

FIG. 5 shows polarization ratio of a circular polarization.

FIG. 6 shows a phase difference dependency of a polarization ratio of a circular polarization.

FIG. 7 shows a configuration of an etching apparatus according to a second embodiment of the present invention.

FIG. 8 is a plan view showing an exemplary configuration of a conductive plate of a patch antenna shown in FIG. 7.

FIGS. 9A and 9B are plan views showing other exemplary configurations of a conductive plate of the patch antenna shown in FIG. 7.

FIG. 10 is a cross sectional view showing part of a configuration of an etching apparatus according to a third embodiment of the present invention.

FIG. 11 shows a two-dimensional configuration of an antenna viewed from the direction of line II-II′ in FIG. 10 and an exemplary configuration of a feeding system thereof.

FIG. 12A is a perspective view showing a configuration of a patch antenna forming the antenna shown in FIG. 10.

FIG. 12B shows coordinate systems.

FIGS. 13A and 13B illustrate principle of radiation of an electromagnetic field by the patch antenna shown in FIG. 12A.

FIG. 14 is a conceptual illustration showing a manner of a magnetic current formed by a patch antenna at a certain moment.

FIG. 15 shows a polarization ratio of a circular polarization.

FIG. 16 shows a phase difference dependency of a polarization ratio of a circular polarization.

FIGS. 17A and 17B show a configuration of a variation of the patch antenna shown in FIGS. 12A and 12B.

FIG. 18 shows a two-dimensional configuration of an antenna used in an etching apparatus according to a fifth embodiment of the present invention and an exemplary configuration of a feeding system thereof.

FIG. 19 is a conceptual illustration showing a manner of a magnetic current formed by a patch antenna at a certain moment.

FIGS. 20A and 20B are conceptual illustrations of a field intensity distribution formed by the antenna shown in FIG. 18.

FIG. 21 shows a configuration of an etching apparatus according to a sixth embodiment of the present invention.

FIG. 22A is a perspective view showing a configuration of the patch antenna shown in FIG. 21.

FIG. 22B shows coordinate systems.

FIGS. 23A and 23B illustrate principle of radiation of an electromagnetic wave by the patch antenna shown in FIG. 21.

FIG. 24 shows a configuration of a variation of the patch antenna shown in FIG. 21.

FIG. 25A shows a configuration of another variation of the patch antenna shown in FIG. 21.

FIG. 25B shows coordinate systems.

FIG. 26 shows a configuration of an etching apparatus according to an eighth embodiment of the present invention.

FIG. 27A is a plan view of a patch 4032 in FIG. 26 viewed from its bottom.

FIG. 27B shows a voltage waveform in x direction in FIG. 27A.

FIG. 27C shows coordinate systems.

FIG. 28 is a plan view showing a variation of the patch.

FIG. 29 is a cross sectional view showing a variation of a patch antenna.

FIG. 30 shows a variation of a patch antenna.

FIG. 31 shows a configuration for generating a circular polarization using a patch antenna shown in FIG. 26.

FIGS. 32A, 32B and 32C illustrate operating principle of a patch antenna with four-point feeding.

FIGS. 33A and 33B are conceptual illustrations showing an electric field distribution formed by the patch antenna shown in FIG. 26.

FIG. 34 illustrates a polarization ratio of a circular polarization.

FIG. 35 shows a phase difference dependency of a polarization ratio of a circular polarization.

FIG. 36 shows a variation of a patch antenna.

FIGS. 37A and 37B show an exemplary configuration of a conventional patch antenna used for a high frequency plasma processing apparatus.

FIG. 37C shows coordinate systems.

FIGS. 38A and 38B illustrate principle of radiation of an electromagnetic field by the patch antenna shown in FIGS. 37A-37C.

FIGS. 39A and 39B are conceptual illustrations of a field intensity distribution formed by the patch antenna shown in FIGS. 37A-37C.

FIG. 40 is a plan view of a dipole antenna conventionally used in a plasma apparatus.

FIG. 41 is a cross sectional view showing an exemplary configuration of an etching apparatus using a conventional high frequency plasma apparatus.

FIG. 42A shows a configuration of a patch antenna shown in FIG. 41.

FIG. 42B shows coordinate systems.

FIGS. 43A and 43B illustrate operating principle of the patch antenna shown in FIG. 41.

### BEST MODES FOR CARRYING OUT THE INVENTION

In the following, embodiments of a first invention of the present invention will be described in detail with reference to the drawings. Here, an example in which a plasma apparatus in accordance with the first invention is applied to an etching apparatus will be described.

### First Embodiment

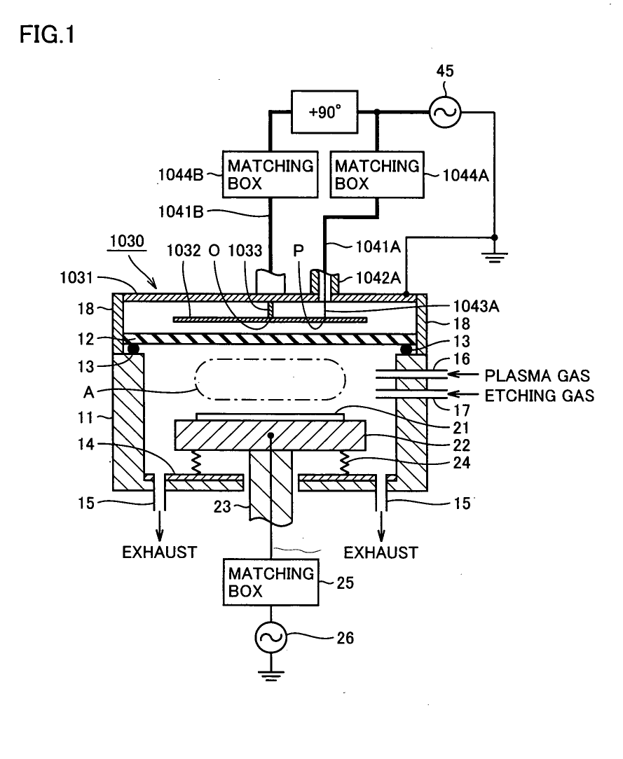

FIG. 1 shows a configuration of an etching apparatus in accordance with a first embodiment of the present invention. FIG. 1 shows some parts in cross section.

The etching apparatus shown in FIG. 1 has a cylindrical processing container 11 that is open at an upper portion. Processing container 11 is formed of a conductive member such as aluminum.

At the upper opening of processing container 11, a dielectric plate 12 formed of quartz glass or ceramic (such as Al₂O₃ or AlN) having a thickness of about 20-30 mm is arranged. At a joint portion between processing container 11 and dielectric plate 12, a sealing member 13 such as an O-ring is interposed, to ensure air-tightness inside processing container 11.

At the bottom of processing container 11, an insulating plate 14 formed of ceramic or the like is provided. Further, an exhaust port 15 is provided penetrating through insulating plate 14 and the bottom of processing container 11 and, by means of a vacuum pump (not shown) communicated with exhaust port 15, the inside of processing container 11 can be evacuated to a desired degree of vacuum.

At upper portion and lower portion of a sidewall of processing container 11, a plasma gas supply nozzle 16 is provided for introducing a plasma gas such as Ar and a processing gas supply nozzle 17 is provided for introducing an etching gas into processing container 11, respectively. Plasma gas supply nozzle 16 and processing gas supply nozzle 17 are formed by a quartz pipe or the like.

In processing container 11, a mounting table 22 is contained, on an upper surface of which a substrate 21 to be etched (the object to be processed) is placed. Mounting table 22 is supported by an elevating shaft 23 passing, with a play, through the bottom of processing container 11 to be freely movable upward and downward. Further, mounting table 22 is connected to a biasing high frequency power supply 26 through a matching box 25. In order to ensure air-tightness of processing container 11, a bellows 24 is provided to surround elevating shaft 23, between mounting table 22 and insulating plate 14.

Above dielectric plate 12, a patch antenna 1030 is arranged for supplying a high frequency wave into processing container 11 through dielectric plate 12. Patch antenna 1030 is separated from processing container 11 by dielectric plate 12, and protected from the plasma generated in processing container 11. Further, a shield member 18 covers peripheries of dielectric plate 12 and patch antenna 1030.

Patch antenna 1030 has a ground plane 1031 made of a grounded conductive plate, and a conductive plate 1032 forming a resonator. Conductive plate 1032 is arranged facing to ground plane 1031 with a prescribed distance, which is maintained by a conductive cylinder 1031 connecting each center of ground plane 1031 and conductive plate 1032. Ground plane 1031, conductive plate 1032 and conductive cylinder 1031 are formed of copper, aluminum or the like. Patch antenna 1030 with such a configuration is arranged such that conductive plate 1032 is located at the bottom side thereof facing to dielectric plate 12.

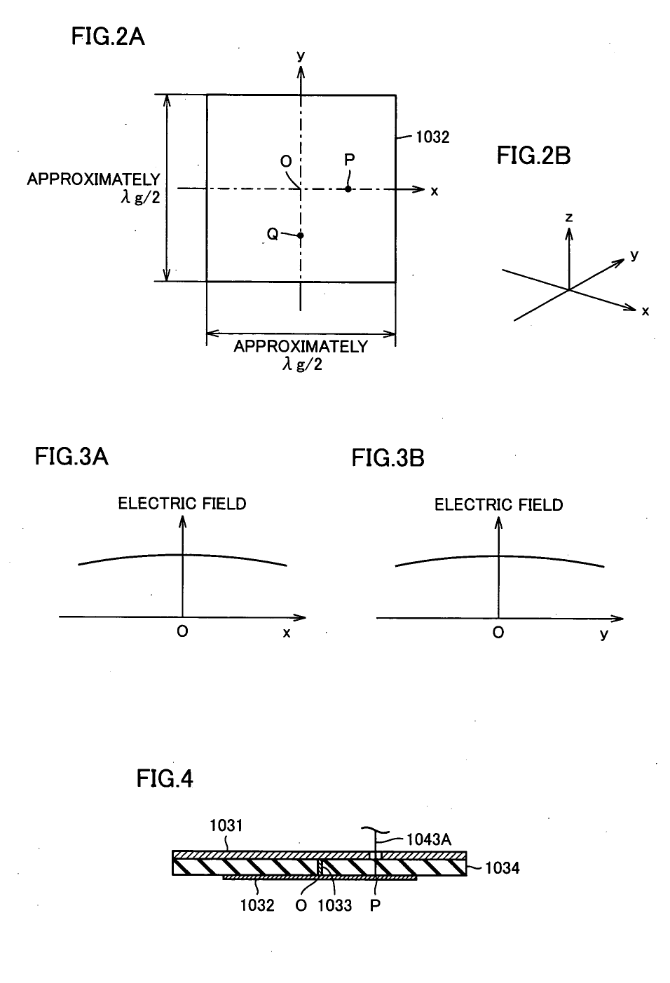

FIGS. 2A and 2B show an exemplary configuration of conductive plate 1032. FIG. 2A is a plan view of conductive plate 1032 in FIG. 1 viewed from the bottom, while FIG. 2B shows coordinate systems. Conductive plate 1032 has a two-dimensional shape of a square with each edge being approximately λg/2. λg means the wavelength of an electromagnetic field in patch antenna 1030. Here, it is assumed that a center O of conductive plate 1032 is at the origin of coordinate systems, and edges of conductive plate 1032 are parallel to x axis and y axis, respectively. In this case, two feeding points P, Q of conductive plate 1032 are provided at two points on x axis and y axis away from center O by approximately equal distances, respectively.

As shown in FIG. 1, two coaxial lines 1041A and 1041B are employed for feeding patch antenna 1030. Outer conductors 1042A and 1042B of coaxial lines 1041A and 1041B are connected to ground plane 1031, while inner conductors (feeding lines) 1043A and 1043B of coaxial lines 1041A and 1041B penetrate through an opening of ground plane 1031 to be connected to conductive plate 1032 at feeding points P, Q thereon, respectively. It is noted that, coaxial line 1041B is longer in electrical length than coaxial line 1041A by 90°.

These coaxial lines 1041A and 1041B are connected to feeding high frequency power supply 45 through matching boxes 1044A and 1044B, respectively. From high frequency power supply 45, a high frequency wave of 100 MHz-8 GHz is output. Additionally, by providing matching boxes 44 for impedance matching, efficiency of power use can be improved.

Next, an operation of the etching apparatus shown in FIG. 1 will be described.

The inside of processing container 11 is evacuated, for example, to a degree of vacuum of about 0.1-10 Pa, with substrate 21 placed on the upper surface of mounting table 22. While maintaining the degree of vacuum, Ar as the plasma gas is supplied from plasma gas supply nozzle 16, and CF₄ as the etching gas is supplied from processing gas supply nozzle 17, with the flow rate thereof being controlled.

In the state where the plasma gas and the etching gas are supplied inside processing container 11, two feeding points P, Q on conductive plate 1032 of patch antenna 1030 are fed at a voltage of an equal amplitude. Here, since coaxial line 1041B is longer in electrical length than axial line 1041A by 90°, the feeding phase of feeding point Q lags behind feeding point P by 90°.

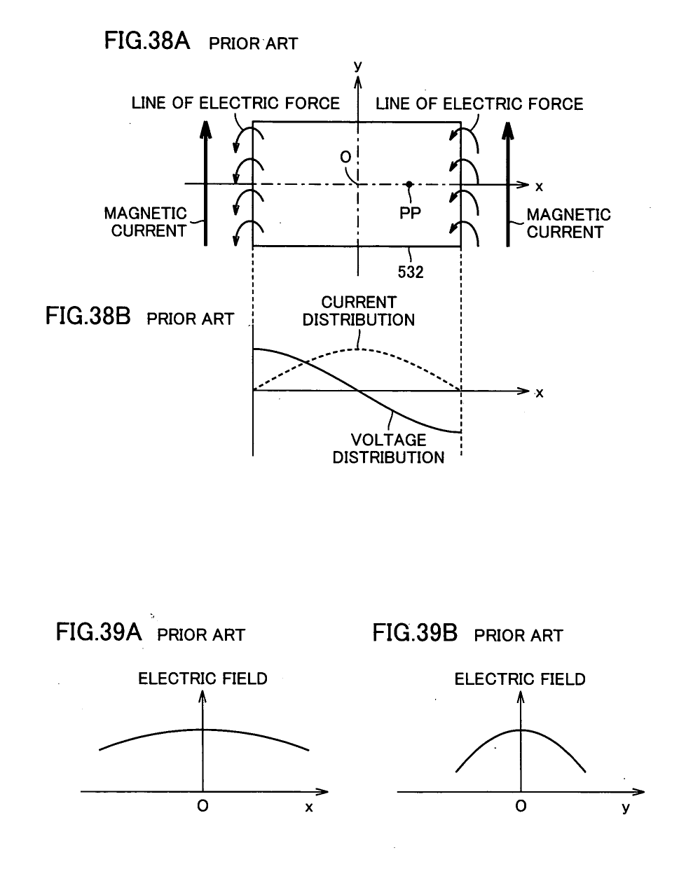

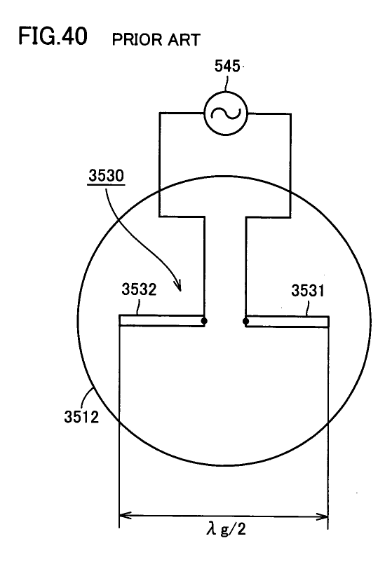

The current supplied to feeding point P oscillates in x axis direction, and emits a high frequency wave of a linear polarization parallel to x axis, according to the same principle as described with reference to FIGS. 38A and 38B. On the other hand, the current supplied to feeding point Q oscillates in y axis direction, and similarly emits a high frequency wave of linear polarization parallel to y axis. It is noted that this linear polarization parallel to y axis lags 90° behind the linear polarization parallel to x axis. These two linear polarizations are equal in amplitude, perpendicular to each other spatially, and having different phases from each other by 90°, and therefore, they become circular polarization. In the positive direction of z axis shown in FIG. 2B, they become right-hand circular polarization.

Thus, the high frequency wave emitted from patch antenna 1030 becomes the circular polarization, passes through dielectric plate 12 and introduced into processing container 11. The high frequency wave forms an electric field in processing container 11 to cause electrolytic dissociation of Ar, and thus generates plasma in upper space A above substrate 21 to be processed.

In this etching apparatus, since negative potential is biased on mounting table 22, therefore ions are extracted from the plasma being generated, and used for the etching process for substrate 21.

FIGS. 3A and 3B are conceptual illustrations of a time-averaged electric field distribution formed by patch antenna 1030. FIG. 3A shows the electric field distribution in xz plane, while FIG. 3B shows the electric field distribution in yz plane. As above, since the high frequency wave emitted by patch antenna 1030 becomes the circular polarization, the electric field distribution thereof will be substantially uniform over xz plane and yz plane, as shown in FIGS. 3A and 3B. As compared to the electric field distribution formed by a conventional patch antenna shown in FIGS. 39A and 39B, it is apparent that the electric field distribution has been improved.

The generation of plasma with the electromagnetic field having such a spatial distribution makes the distribution of plasma uniform, therefore the etching process can be performed at uniform speed over the entire substrate 21.

The two-dimensional shape of conductive plate 1032 may be a 90° rotationally symmetrical shape (a shape that overlaps when rotated by 90° around a central axis of conductive plate 1032) such as a circle, in addition to the square form shown in FIG. 2A. It is noted that when employing a circle, diameter thereof may be 1.17 λg/2.

Further, the two-dimensional shape of conductive plate 1032 may be a shape in which its lengths in two directions perpendicular to each other viewed from its center O are different, e.g., a rectangle. In this case, the feeding phase difference between two feeding points P, Q may not be 90°, and it is adjusted by the lengths in the two directions described above.

Still further, in the etching apparatus shown in FIG. 1, it has been described that the right-hand circular polarization is emitted in the positive direction of z axis shown in FIG. 2B. To emit the left-hand circular polarization, coaxial line 1041A may be set to have an electrical length longer than coaxial line 1041B by 90°, conversely.

Still further, ground plane 1031 and conductive plate 1032 forming the patch antenna may be formed at two opposing surfaces of dielectric plate 34 made of ceramic or the like, as shown in FIG. 4. Thus, the patch antenna can be made smaller.

Still further, the high frequency wave emitted by the patch antenna is not necessarily a perfect circular polarization. Given that the polarization ratio of a circular polarization of which length of long axis is 2a and that of short axis is 2b as shown in FIG. 5 is b/a (×100)%, generation of the circular polarization with a polarization ratio of at least 50%, preferably at least 70% results in the improvement of the plasma distribution.

Here, a method for adjusting the polarization ratio of a circular polarization is described briefly.

First, when two linear polarizations perpendicular to each other are out of phase to each other by 90° but with different amplitude values, given that the two linear polarizations are expressed as a sin (ωt+π/2), b sin (ωt), then the polarization ratio is simply determined by the amplitude value ratio b/a (×100)%. Therefore, in order to obtain the polarization ratio of at least 70%, the amplitude value ratio may only be set to at least 70%.

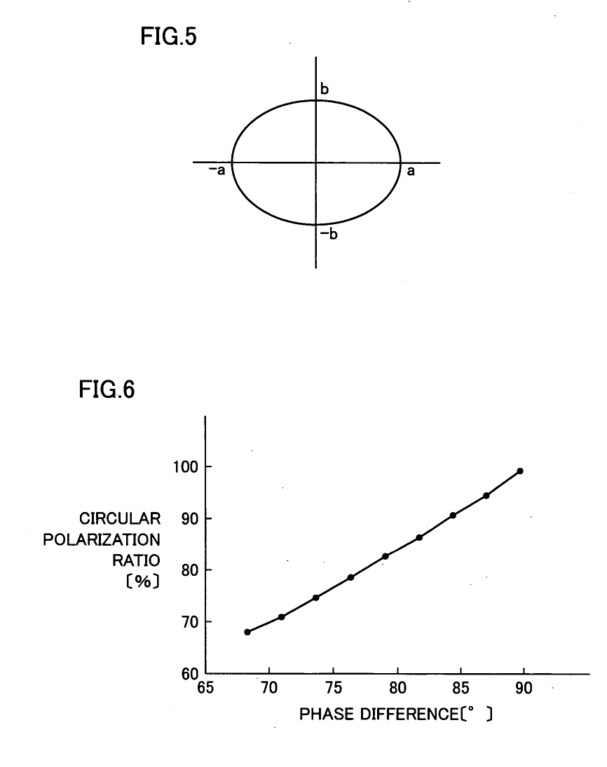

Additionally, when two linear polarizations perpendicular to each other are equal in amplitude value but with a phase difference other than 90°, given that the two linear polarizations are expressed as sin (ωt−θ), sin (ωt), then the phase difference dependency of the polarization ratio when the phase difference θ takes on the value in the vicinity of 90° will be as shown in FIG. 6. Therefore, in order to obtain the polarization ratio of at least 70%, the phase difference θ may only be adjusted to approximately 70°-110°.

### Second Embodiment

Next, a second embodiment of the present invention will be described. In the first embodiment, though it has been described to emit a circular polarization by feeding patch antenna 1030 at two points thereof, it is also possible to emit a circular polarization by feeding at only one point.

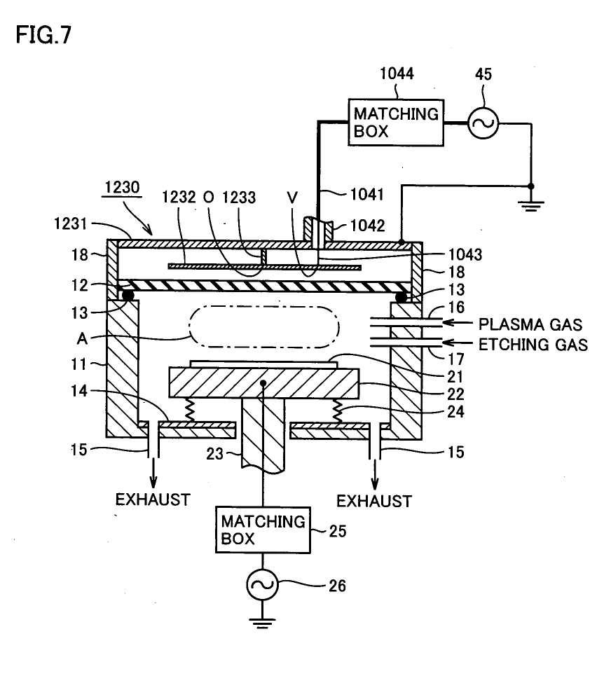

FIG. 7 shows a configuration of an etching apparatus according to the second embodiment of the present invention. In the figure, portions similar to those of FIG. 1 will be denoted by the same reference characters and description thereof will appropriately be omitted.

A patch antenna 1230 shown in FIG. 7 includes a ground plane 1231, a conductive plate 1232 forming a resonator, and a conductive cylinder 233 connecting a center O of conductive plate 1232 to ground plane 1231.

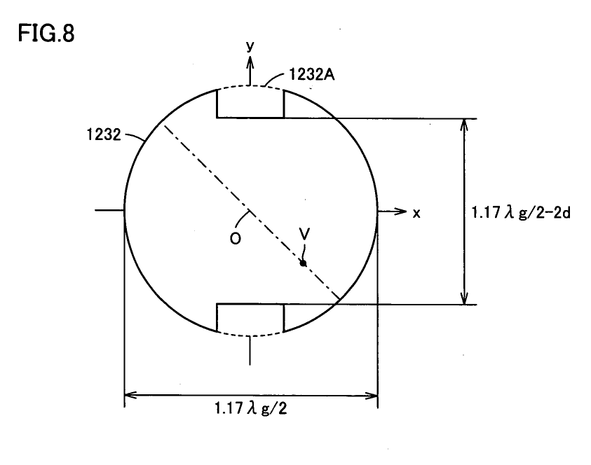

FIG. 8 is a plan view showing an exemplary configuration of conductive plate 1232, which illustrates a two-dimensional shape of conductive plate 1232 in FIG. 7 viewed from the bottom. In this figure, portion similar to those of FIGS. 2A, 2B will be denoted by the same reference characters and description thereof will appropriately be omitted.

The two-dimensional shape of conductive plate 1232 corresponds to circle 1232A of which peripheral regions are cut out partially. More specifically, two regions in the vicinity where circumference and y axis cross are cut out in quadrangular forms. The area to be the cut out may be approximately 3% of circle 1232A. It is assumed that the length in x direction of conductive plate 1232 is 1.17×λg/2, while the length in y axis direction thereof is 1.17×λg/2−2 d.

A feeding point V is provided at one point on the straight line that crosses x axis and y axis at the angle of 45°. As shown in FIG. 7, to feeding point V, an inner conductor 1043 of coaxial line 1041 connected to high frequency power supply 45 is connected.

The current supplied from high frequency power supply 45 to feeding point V of conductive plate 1232 will flow in x axis direction and y axis direction, independently. Here, since y axis direction is shorter than 1.17×λg/2 by 2 d, the permittivity in the electric magnetic field becomes greater, causing the phase lag of the current passing in y axis direction. By setting the value of 2 d and the length of the cut out portion such that the phase lag of 90° can be obtained, the current can be passed in x and y axis directions of conductive plate 1232 with the phase lag of 90°. Thus, the circular polarization can be emitted from patch antenna 1230.

While it has been described that feeding point V is provided at one point on a straight line crossing x axis and y axis at the angle of 45°, it may be provided at one point in a direction between x axis direction and y axis direction, if a perfectly circular polarization is not required.

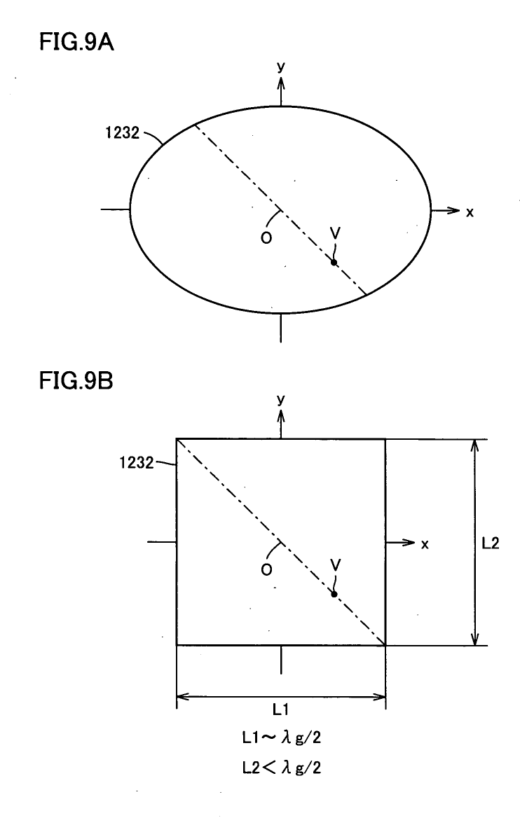

The two-dimensional shape of conductive plate 1232 is not limited to the shape shown in FIG. 8, and it may be at least the shape in which two directions perpendicular to each other viewed from center O of conductive plate 1232 are different. Accordingly, it may be an ellipse as shown in FIG. 9A. Additionally, as shown in FIG. 9B, it may be a quadrangle in which the length of long edge L1 is approximately λg/2, while the length of short edge L2 is less than approximately λg/2.

Further, similarly to the first embodiment, a dielectric plate formed of ceramic or the like as shown in FIG. 4 may be provided between ground plane 1231 and conductive plate 1232. Thus, the patch antenna can be made smaller.

In the foregoing, though an example where the plasma apparatus according to the first invention is applied to an etching apparatus has been described, it is needless to say that it may be applied to other plasma apparatuses such as a plasma CVD apparatus.

As described above, the plasma apparatus according to the first invention uses the antenna having the conductive plate forming the resonator and the ground plane arranged facing to the conductive plate, to make the high frequency wave emitted from the antenna to be a circular polarization. Thus, the spatial distribution of the electromagnetic field in the processing container, and therefore the distribution of plasma, becomes uniform as compared to the conventional manner.

In the following, referring to the figures, embodiment according to a second invention of the present invention will be described in detail. Here, an example will be described where a plasma processing apparatus according to the second invention is applied to an etching apparatus.

### Third Embodiment

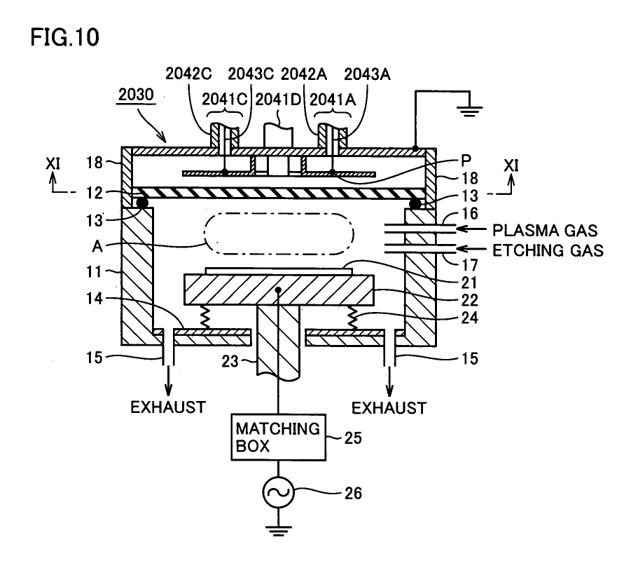

FIG. 10 is a cross-sectional view showing part of the configuration of an etching apparatus according to a third embodiment of the present invention.

The etching apparatus shown in FIG. 10 has a cylindrical processing container 11 that is open at an upper portion. Processing container 11 is formed of a conductive member such as aluminum.

At the upper opening of processing container 11, a dielectric plate 12 formed of quartz glass or ceramic (for example, Al₂O₃ or AlN) having a thickness of about 20-30 mm is arranged. At a joint portion between processing container 11 and dielectric plate 12, a sealing member 13 such as an O-ring is interposed, to ensure air-tightness inside processing container 11.

At the bottom of processing container 11, an insulating plate 14 formed of ceramic or the like is provided. Further, an exhaust port 15 is provided penetrating through insulating plate 14 and the bottom of processing container 11 and, by means of a vacuum pump (not shown) communicated with exhaust port 15, the inside of processing container 11 can be evacuated to a desired degree of vacuum.

At upper portion and lower portion of a sidewall of processing container 11, a plasma gas supply nozzle 16 is provided for introducing a plasma gas such as Ar and a processing gas supply nozzle 17 is provided for introducing an etching gas into processing container 11, respectively. Plasma gas supply nozzle 16 and processing gas supply nozzle 17 are formed by a quartz pipe or the like.

In processing container 11, a mounting table 22 is contained, on an upper surface (a mounting surface) of which a substrate 21 to be etched (the object to be processed) is placed. Mounting table 22 is supported by an elevating shaft 23 passing, with a play, through the bottom of processing container 11 to be freely movable upward and downward. Further, mounting table 22 is connected to a biasing high frequency power supply 26 through a matching box 25. Output frequency of high frequency power supply 26 is a prescribed frequency within the range of several hundred kHz to ten and several MHz. In order to ensure air-tightness of processing container 11, a bellows 24 is-provided to surround elevating shaft 23, between mounting table 21 and insulating plate 14.

Above dielectric plate 12, an antenna 2030 is arranged for supplying an electromagnetic field of a high frequency wave into processing container 11 through dielectric plate 12. Antenna 2030 is separated from processing container 11 by dielectric plate 12, and protected from the plasma generated in processing container 11. Further, a shield member 18 covers peripheries of dielectric plate 12 and antenna 2030 to prevent leakage of the electromagnetic field emitted from antenna 2030 to the outside of the etching apparatus.

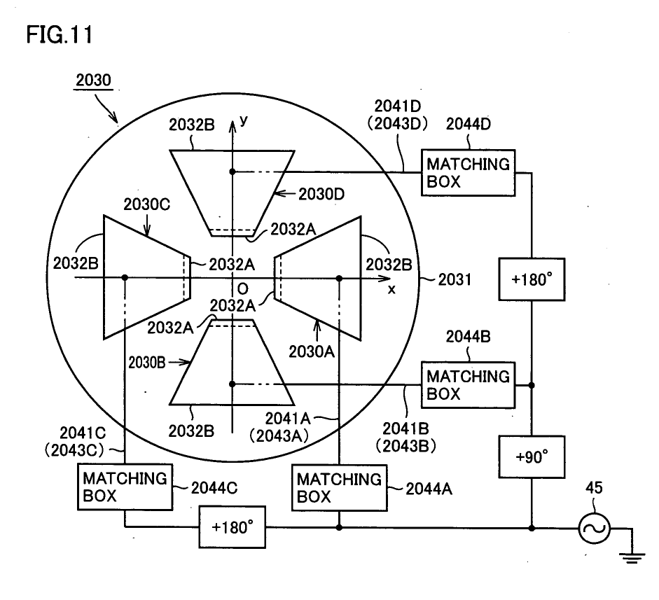

FIG. 11 shows a two-dimensional configuration of antenna 2030 in FIG. 10 viewed from the bottom, and an exemplary configuration of feeding system thereof. Antenna 2030 is formed by combining four monopole patch antennas 2030A, 2030B, 2030C, and 2030D each having a conductive plate 2032 having a trapezoid shape in two dimensions. Here, of two parallel edges of conductive plate 2032, the shorter edge and the longer edge are denoted as ends 2032A and 2032B, respectively. Then, patch antennas 2030A-2032D are arranged with equal intervals around center O of antenna 2030, with each end 2032A directed inward, and end 2032B directed outward. As shown in FIG. 10, it is arranged such that conductive plate 2032 is located at the bottom facing to dielectric plate 12.

For feeding patch antennas 2030A-2030D, coaxial lines 2041A, 2041B, 2041C and 2041D are employed, respectively.

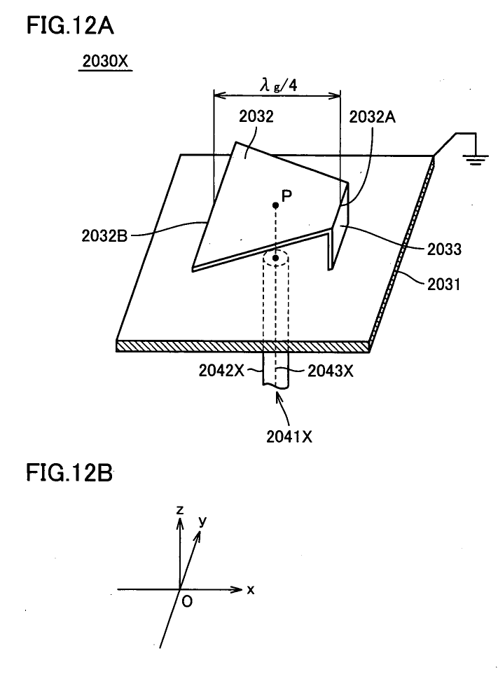

Four patch antennas 2030A-2030D forming antenna 2030 have identical configurations. Here, patch antennas 2030A-2030D are collectively denoted as patch antenna 2030X (X refers to A, B, C and D). Further, coaxial lines 2041A-2041D are collectively denoted as coaxial line 2041X (X refers to A, B, C, and D). FIGS. 12A and 12B show a configuration of patch antenna 2030X. Here, FIG. 12A is a perspective view, while FIG. 12B shows coordinate systems.

As shown in FIG. 12A, patch antenna 2030X includes a ground plane 2031 formed of a grounded conductive plate, a conductive plate 2032 forming a resonator and having a two-dimensional shape of a trapezoid, and a conductive member 2033 connecting end 2032A of conductive plate 2032 to ground plane 2031.

For ease of description, coordinate systems are defined as follows x axis is set in the direction of the height of the trapezoid of conductive plate 2032, y axis is set in a direction parallel to the parallel two edges of the trapezoid, and z axis is set in the positive direction of conductive plate 2032 leading from ground plane 2031 toward conductive plate 2032.

Conductive plate 2032 forming a resonator is arranged facing to and parallel to ground plane 2031. Conductive plate 2032 has a shape of trapezoid as described above, and when the wavelength of the electromagnetic field between conductive plate 2032 and ground plane 2031 is given as λg, then the height of the trapezoid (specifically, the length in x axis direction perpendicular to end 2032A) is set to approximately λg/4. Additionally, the length of end 32B of conductive plate 2032 is desirably less than approximately λg/2.

Conductive member 2033 connecting end 2032A of conductive plate 2032 to ground plane 2031 is a plate-like member provided perpendicular to ground plane 2031. The length of conductive member 2033 in y axis direction is equal to that of end 2032A of conductive plate 2032, and the length in z axis direction (i.e., height) is desirably 5-50 mm, approximately.

End 2032A of conductive plate 2032 is short-circuited to ground plane 2031 by conductive member 2033, and therefore the potential of end 2032A is fixed to 0 (zero) even when being fed from coaxial line 2041X. Hence, end 2032A is referred to as fixed end 2032A. On the other hand, end 32B opposing to fixed end 2032A with a distance of λg/4 is open, and hence it is referred to as open end 32B.

It is noted that ground plane 2031 is a member shared by patch antennas 2030A-2030D, and have a circular shape similarly to dielectric plate 12.

Ground plane 2031, conductive plate 2032 and conductive member 2033 are formed of copper, aluminum or the like.

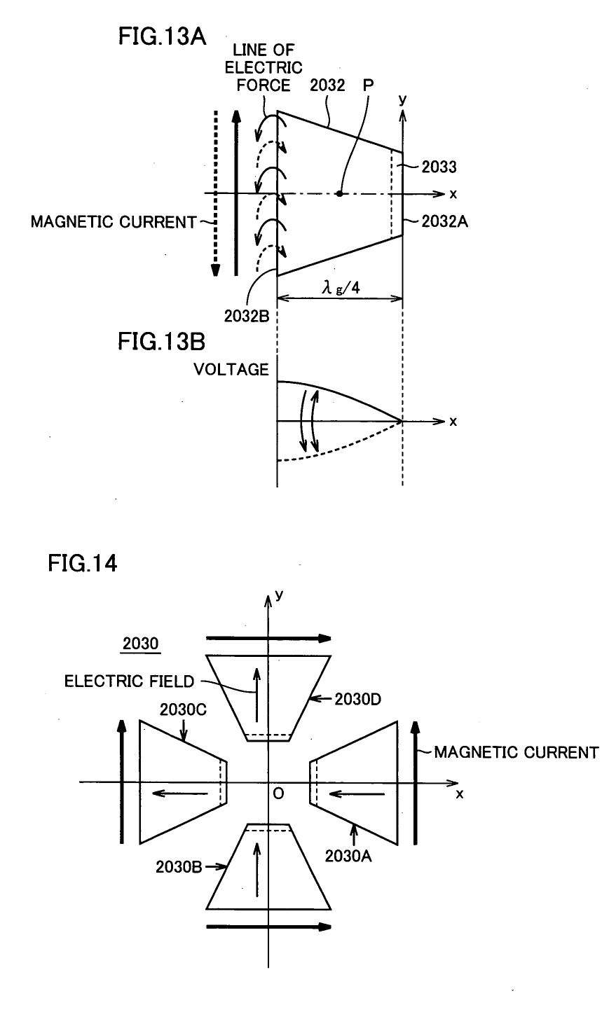

Here, the principle of radiation of electromagnetic field by patch antenna 2030X is described. FIGS. 13A and 13B are illustrations therefor. FIG. 13A shows conductive plate 2032, while FIG. 13B shows the voltage distribution in x axis direction in conductive plate 2032.

The potential of fixed end 2032A of conductive plate 2032 is fixed to 0 (zero), and that the length of conductive plate 2032 in x axis direction is λg/4. Therefore, the current supplied from high frequency power supply 45 to conductive plate 2032 behaves as if the length of conductive plate 2032 in x axis direction is λg/2, oscillating in x axis direction to be a standing wave. Here, the voltage waveform repeats the change shown by a solid line and a dotted line in FIG. 13B.

When the voltage at open end 32B of conductive plate 2032 is positive, the line of electric force is directed from conductive plate 2032 toward ground plane 2031 as indicated by the solid line in FIG. 13A, and when the voltage at open end 32B of conductive plate 2032 is negative, the line of electric force is directed from ground plane 2031 toward conductive plate 2032 as indicated by the dotted line in FIG. 13B. The direction of the line of electric force is the same as the direction of the displacement current, and therefore a magnetic current repeating the change shown by the solid line and the dotted line in FIG. 13A is generated along open end 2032B and parallel to y axis. Since an electromagnetic field is emitted having the magnetic current as a wave source, the electromagnetic field becomes a linear polarization parallel to x axis.

Patch antenna 2030X has an excellent radiation efficiency of the electromagnetic field due to its long open end 32B that functions as a magnetic current forming portion. From the view point of the radiation efficiency, the length of conductive plate 2032 in y axis direction should be longer. In order to decrease the effect of the magnetic current formed in the side face of patch antenna 2030X parallel to x axis, the length of conductive plate 2032 in y axis direction is preferably set to at least λg/8, approximately.

On the other hand, as shown in FIG. 12A, outer conductor 2042X of coaxial line 2041X (X refers to A, B, C, and D) is connected to ground plane 2031, while inner conductor (feeding line) 43X of coaxial line 2041X (X refers to A, B, C, and D) penetrates through the opening of ground plane 2031 to be connected to feeding point P on conductive plate 2032. Feeding point P is provided at a point away from fixed end 2032A of conductive plate 2032. Considering impedance matching and the like, it is desirable to set feeding point P near the center of conductive plate 2032.

As shown in FIG. 11, coaxial lines 2041A-2041D respectively connected to patch antennas 2030A-2030D are connected to feeding high frequency power supply 45. It is noted that the electrical lengths of coaxial lines 2041A-2041D are each longer by multiples of 90°, with reference to coaxial line 2041A. Specifically, when the electrical length of coaxial line 2041A is θ, electrical length of coaxial lines 2041B, 2041C and 2041D are θ+90°, θ+180°, and θ+270°, respectively. Thus, feedings to patch antennas 2030A-2030D are performed with a phase difference of 90° each. Here, patch antennas 2030A-2030D are fed with equal power.

It is noted that output frequency of feeding high frequency power supply 45 is a prescribed frequency in a range of about 100 MHz-8 GHz. Additionally, by interposing matching boxes 2044A, 2044B, 2044C, and 2044D for impedance matching in coaxial lines 2041A-2041D, respectively, efficiency of power use can be improved.

Next, an operation of the etching apparatus shown in FIG. 10 will be described.

The inside of processing container 11 is evacuated, for example, to a, degree of vacuum of about 0.01-10 Pa, with substrate 21 placed on the upper surface of mounting table 22. While maintaining the degree of vacuum, Ar as the plasma gas is supplied from plasma gas supply nozzle 16, and CF₄ as the etching gas is supplied from processing gas supply nozzle 17, with the flow rate thereof being controlled.

In the state where the plasma gas and the etching gas are supplied inside processing container 11, feeding from high frequency power supply 45 to antenna 2030 is initiated. At this time, patch antennas 2030A-2030D are fed with a phase difference of 90° each.

FIG. 14 is a conceptual illustration showing the manner of magnetic current formed by patch antennas 2030A-2030D at a certain moment. The feeding phase difference between patch antennas 2030A and 2030C is 180°, therefore patch antennas 2030A and 2030C form magnetic currents in the same direction parallel to y axis. Thus, patch antennas 2030A and 2030C each emit a linear polarization of the same phase parallel to x axis. Similarly, the feeding phase difference between patch antennas 2030B and 2030D is 180°, therefore patch antennas 2030B and 2030D each emit a linear polarization of the same phase parallel to y axis. In addition, since the feeding phase difference between patch antennas 2030A and 2030B (or between 2030C and 2030D) is 90° C., the phase difference between the linear polarization in x axis direction and that of y axis direction is 90°. These two linear polarizations are equal in amplitude, perpendicular to each other spatially, and have a the phase difference of 90°, and therefore a circular polarization is formed.

Thus, the electromagnetic field emitted from patch antenna 2030 forms the circular polarization, passes through dielectric plate 12 and introduced into processing container 11. Then, formation of electric field in processing container 11 causes electrolytic dissociation of Ar, and thus generates plasma in upper space A above substrate 21 to be processed. The plasma diffuses into processing container 11, and has its energy and anisotropy controlled by a bias voltage applied to mounting table 22, and utilized for the etching process.

The field intensity distribution by linear polarizations emitted by patch antennas 2030A and 2030C (or patch antennas 2030B and 2030D) is biased as in FIGS. 39A and 39B. However, by forming a circular polarization to rotate the electric field around an axis perpendicular to mounting table 22, the plasma distribution generated by this electric field also rotates, and therefore the etching process can be performed uniform when averaged by time than the conventional manner.

Though an example has been described where four patch antennas 2030A-2030D are employed to make the electromagnetic field to be a circular polarization, the number of patch antenna 2030X for forming a circular polarization may be at least two.

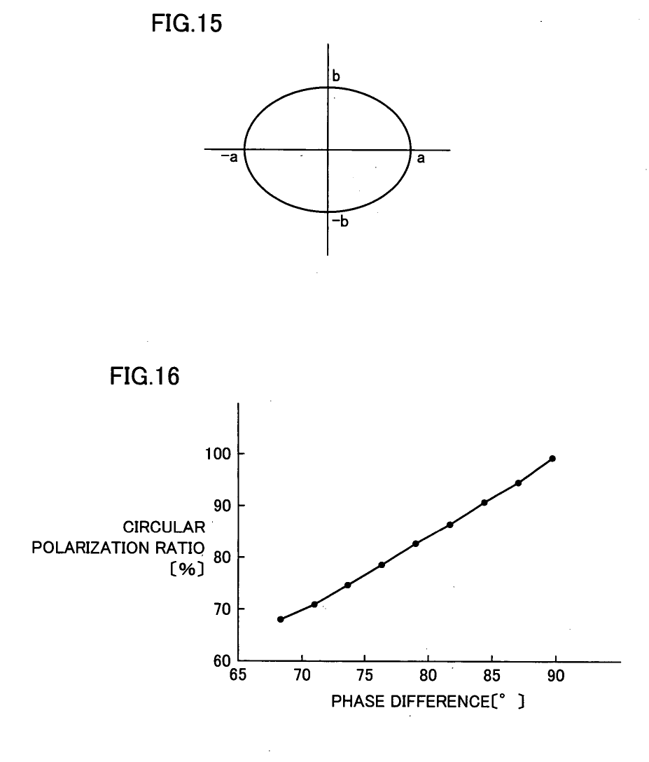

Additionally, the electromagnetic field supplied to processing container 11 is not necessarily a perfectly circular polarization. Given that the polarization ratio of a circular polarization of which length of long axis is 2a and that of short axis is 2b as shown in FIG. 15 is b/a (×100)%, generation of circular polarization with a polarization ratio of at least 50%, preferably at least 70% results in the improvement of the uniformity over a plane in processing.

Here, a method for adjusting the polarization ratio of a circular polarization is described briefly.

First, when two linear polarizations perpendicular to each other are out of phase to each other by 90° but with different amplitude values, given that the two linear polarizations are expressed as a sin(ωt+π/2), b sin(ωt), then the polarization ratio is simply determined by the amplitude value ratio b/a (×100)%. Therefore, in order to obtain the polarization ratio of at least 70%, the amplitude value ratio may only be set to at least 70%.

Additionally, when two linear polarizations perpendicular to each other are equal in amplitude value but with a phase difference other than 90°, given that the two linear polarizations are expressed as sin(ωt−θ), sin(ωt), then the phase difference dependency of the polarization ratio when the phase difference θ takes on the value in the vicinity of 90° will be as shown in FIG. 16. Therefore, in order to obtain the polarization ratio of at least 70%, the phase difference θ may only be adjusted to approximately 70°-110°.

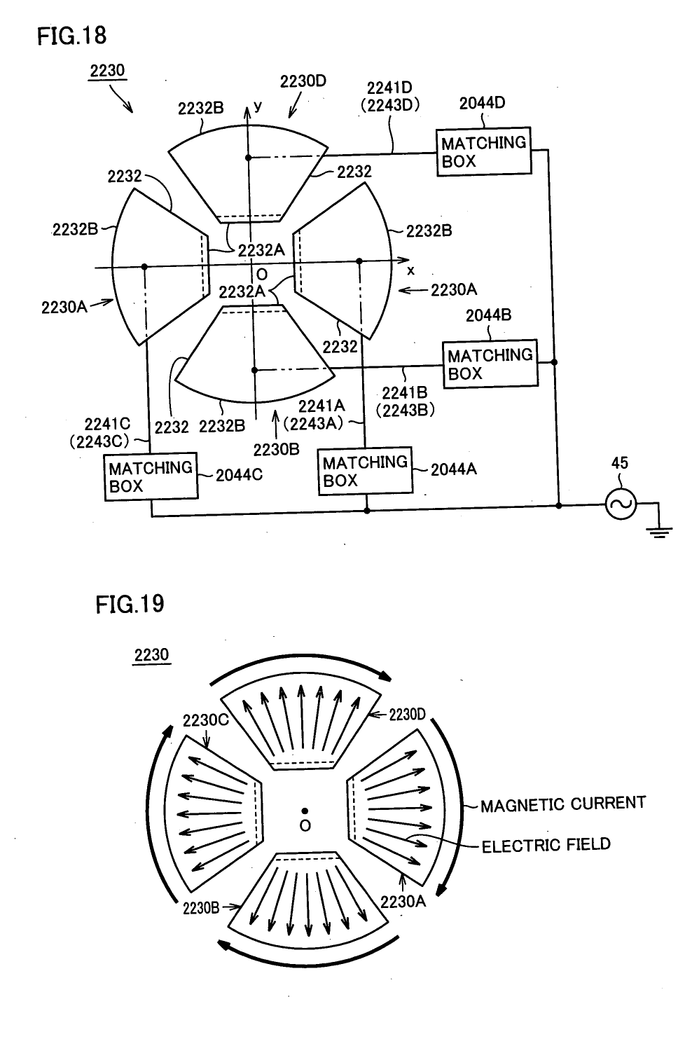

Though patch antenna 2030X shown in FIG. 12A has a two-dimensional shape of a trapezoid, the shape thereof is only required to have straight fixed end 2032A and a length of approximately λg/4 in x axis direction perpendicular to fixed end 2032A. Accordingly, it may be a quadrangular or a semi-circular shape two-dimensionally. Still further, as shown in FIG. 18 that follows, a patch antenna in which open end of conductive plate is circular arc may be employed. Still further, both of the open end and the fixed end may be circular arc.

Still further, a dielectric plate may be arranged between ground plane 2031 and conductive plate 2032 forming patch antenna 2030X. Thus, the wavelength λg of the electromagnetic field between conductive plate 2032 and ground plane 2031 become shorter, therefore patch antenna 2030X can be made smaller.

### Fourth Embodiment

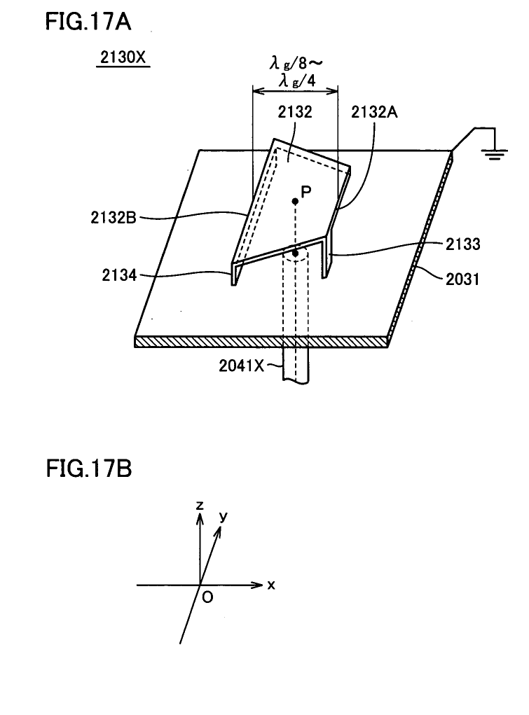

Next, a variation of patch antenna 2030X shown in FIG. 12A will be described. FIGS. 17A and 17B show a configuration of the variation. FIG. 17A is a perspective view of patch antenna 2130X, while FIG. 17B shows coordinate systems. In these figures, portions similar to those of FIGS. 12A and 12B are denoted by similar reference characters, and description thereof will appropriately be omitted.

A patch antenna 2130X shown in FIG. 17A has, in the configuration of patch antenna 2030X shown in FIG. 12A, open end 2032B of conductive plate 2032 folded in the direction of ground plane 2031, and conductive member 2033 cut out partially to be a narrow shape.

In the following, description will be made in detail. In patch antenna 2130X, the length of conductive plate 2132 forming a resonator in x axis direction is at least approximately λg/8 and less than approximately λg/4.

Additionally, to conductive plate 2132, a conductive plate 2134 extending from open end 2132B toward ground plane 2031 is connected. The length of conductive plate 2134 in y axis direction is the same as that of conductive plate 2132, while the length thereof in z axis direction is shorter than that of conductive member 2133. Accordingly, the tip of conductive plate 2134 does not contact ground plane 2031. Further, conductive plate 2134 is made of the same material as conductive plate 2132 and the like, i.e., copper or aluminum.

Still further, conductive member 2133 connecting fixed end 2132A of conductive plate 2132 to ground plane is shorter in y axis direction than conductive plate 2132.

When patch antenna 2130X with such a configuration is fed, large capacitance is formed between conductive plate 2134 and ground plane 2031. Additionally, large inductance is formed in the narrow conductive member 2133. By designing the apparatus such that these capacitance and inductance compensate each other, the length of conductive plate 2132 in x axis direction can be shortened to approximately λg/8, and the patch antenna can be made smaller. Conversely, since it allows to emit an electromagnetic field of a lower frequency wave by the same antenna size, the degree of freedom in designing the etching apparatus, of which antenna size has been limited by the diameter of processing container 11, can be increased.

On the other hand, patch antenna 2130X is capable of emitting the electromagnetic field equivalent to those emitted by patch antenna 2030X shown in FIG. 12A. Therefore, patch antenna 2130X can be used to form an antenna for supplying the electromagnetic field to achieve the uniformity over a plane, which is equivalent to those achieved by the process with the etching apparatus according to the third embodiment.

### Fifth Embodiment

While the etching apparatus according to the third embodiment employs a plurality of patch antennas 2030X to make the electromagnetic field supplied into processing container 11 to be a circular polarization, an etching apparatus according to a third embodiment makes the electromagnetic field to be a substantial TM01 mode. The configuration of the etching apparatus according to the third embodiment that corresponds to FIG. 10 is similar to that of the third embodiment, and therefore description thereof is omitted.

FIG. 18 shows a two-dimensional configuration of an antenna 2230 used in the etching apparatus according to the fifth embodiment of the present invention, and an exemplary configuration of its feeding system. In the figure, portions similar to those of FIG. 11 are denoted by the similar reference characters, and description thereof will appropriately be omitted.

Antenna 2230 is formed by combining four monopole patch antennas 2230A-2230D. Though patch antennas 2230A-2230D as a whole are configured similarly to patch antenna 2030X shown in FIG. 12A, the difference between them is that open end 2232B of conductive plate 2232 forming a resonator is formed in a circular arc. Preferably, when four patch antennas 2230A-2230D are arranged with equal intervals around center O of antenna 2230 with each open end 2232B directed outward, the line connecting each open end 2232B forms a substantial circle. Here, the length from fixed end 2232A to open end 2232B of each patch antenna 2230A-2230D are approximately (1.17±0.05)×λg/4 on x axis or on y axis.

For feeding patch antennas 2230A-2230D, coaxial lines 2241A, 2241B, 2241C, and 2241D are employed, respectively. The electrical lengths of coaxial lines 2241A-2241D are all equal, which is different from coaxial lines 2041A-2041D. Accordingly, patch antennas 2230A-2230D are all fed with the same phase.

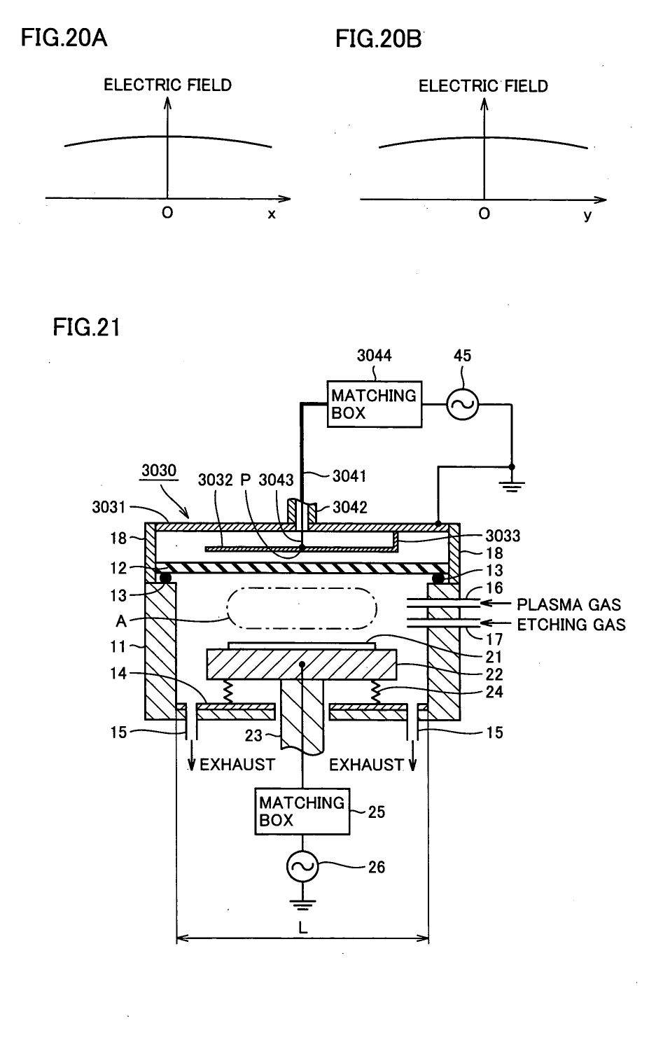

FIG. 19 is a conceptual illustration showing the manner of magnetic currents formed by patch antennas 2230A-2230D at a certain moment. As patch antennas 2230A-2230D are fed with the same phase, patch antennas 2230A-2230D form magnetic currents in the same direction along their respective open ends 2232B. Since the magnetic currents are formed on the same circumference, the electric field of an electromagnetic field having the magnetic currents as the wave source is distributed approximately radially around center O of antenna 2230. Here, the mode of the electromagnetic field showing such an electric field distribution is referred to as a substantial TM01 mode. The field intensity distribution on xz plane and on yz plane of the substantial TM01 mode will be approximately uniform as shown in FIGS. 20A and 20B, respectively. As compared to the field intensity distribution formed by the single patch antenna that operates like a dipole as shown in FIG. 14, it is found that the field intensity distribution is improved by antenna 2230 shown in FIG. 18.

By generating plasma by the electric field having such a uniform spatial distribution as shown in FIGS. 20A and 20B, the plasma distribution can be made uniform. This enables to perform etching process at uniform speed over the entire substrate 21.

Though the example has been described where four patch antennas 2230A-2230D are employed to make the electromagnetic field to be the substantial TM01 mode, the substantial TM01 mode can be formed with at least two patch antennas if they each have open end 2232B of conductive plate 2232 of a circular arc. Further, an antenna may be formed with at least three patch antennas 2030X having straight open end 32B of conductive plate 2032 as shown in FIG. 12A.

Still further, in order to approximate the electromagnetic field emitted by antenna 2230 shown in FIG. 19 to a perfect TM01 mode, the intervals between each of patch antennas 2230A-2230D may be set shorter.

Still further, a dielectric plate may be arranged between ground plane 2031 and conductive plate 2232 forming patch antennas 2230A-2230D.

Still further, a patch antenna having the configuration as shown in FIG. 17A may be employed. In this case, open end 2132B of conductive plate 2132 may be shaped in a circular arc. Here, the length from fixed end 2132A to open end 2232B is set to approximately at least 1.17×λg/8, and approximately less than 1.7×λg/4 on x axis or on y axis.

In the foregoing, though the example has been described where the plasma processing apparatus according to the second invention is applied to an etching apparatus, it is needless to say that it can be applied to other plasma processing apparatus such as a plasma CVD apparatus.

As described above, in the plasma processing apparatus according to the second invention, the antenna formed by a plurality of monopole antennas is used to supply the electromagnetic field of the circular polarization into the processing container. The circular polarization rotates the electromagnetic field around the axis perpendicular to the mounting surface of the mounting table, to rotates the plasma distribution generated by this electromagnetic field as well. Thus, the plasma process can be performed uniform when averaged by time as compared to the conventional manner.

Further, the antenna formed by a plurality of monopole antennas is used to supply the electromagnetic field of substantial TM01 mode into processing container. The electric field in the substantial TM01 mode is distributed approximately radially around the axis perpendicular to the mounting surface of the mounting table, and therefore the uniformity of the plasma distribution on a plane that is parallel to the mounting surface can be improved. Accordingly, the plasma process can be performed uniform as compared to the conventional manner.

Still further, by using the patch antenna as a monopole antenna, the radiation efficiency of the electromagnetic field can be improved.

In the following, referring to the figures, embodiments according to a third invention of the present invention will be described in detail. Here, an example will be described where a plasma apparatus according to the third invention is applied to an etching apparatus.

### Sixth Embodiment

FIG. 21 shows a configuration of an etching apparatus according to a sixth embodiment of the present invention. FIG. 21 shows a cross section for part of the configuration.

The etching apparatus shown in FIG. 21 has a cylindrical processing container 11 that is open at an upper portion. Processing container 11 is formed of a conductive member such as aluminum.

At the upper opening of processing container 11, a dielectric plate 12 formed of quartz glass or ceramic (for example, Al₂O₃, AlN or the like) having a thickness of about 20-30 mm is arranged. At a joint portion between processing container 11 and dielectric plate 12, a sealing member 13 such as an O-ring is interposed, to ensure air-tightness inside processing container 11.

At the bottom of processing container 11, an insulating plate 14 formed of ceramic or the like is provided. Further, an exhaust port 15 is provided penetrating through insulating plate 14 and the bottom of processing container 11 and, by means of a vacuum pump (not shown) communicated with exhaust port 15, the inside of processing container 11 can be evacuated to a desired degree of vacuum.

At upper portion and lower portion of a sidewall of processing container 11, a plasma gas supply nozzle 16 is provided for introducing a plasma gas such as Ar and a processing gas supply nozzle 17 is provided for introducing an etching gas into processing container 11, respectively. Plasma gas supply nozzle 16 and processing gas supply nozzle 17 are formed by a quartz pipe or the like.

In processing container 11, a mounting table 22 is contained, on an upper surface of which a substrate 21, such as a wafer to be etched, is placed. Mounting table 22 is supported by an elevating shaft 23 passing, with a play, through the bottom of processing container 11 to be freely movable upward and downward. Further, mounting table 22 is connected to a biasing high frequency power supply 26 through a matching box 25. Output frequency of high frequency power supply 26 is about several hundred kHz to ten and several MHz. In order to ensure air-tightness of processing container 11, a bellows 24 is provided to surround elevating shaft 23, between mounting table 22 and insulating plate 14.

Above dielectric plate 12, a patch antenna 3030 is arranged for supplying an electromagnetic field of a high frequency wave into processing container 11 through dielectric plate 12. Patch antenna 3030 is separated from processing container 11 by dielectric plate 12, and protected from the plasma generated in processing container 11. Further, a shield member 18 covers peripheries of dielectric plate 12 and patch antenna 3030 to prevent leakage of the high frequency wave to the outside of the etching apparatus.

Coaxial line 3041 is used for feeding patch antenna 3030. Coaxial line 3041 is connected to feeding high frequency power supply 45 through matching box 3044. Output frequency of high frequency power supply 45 is approximately 100 MHz-8 GHz. By providing matching box 3044 for impedance matching, efficiency of power use can be improved.

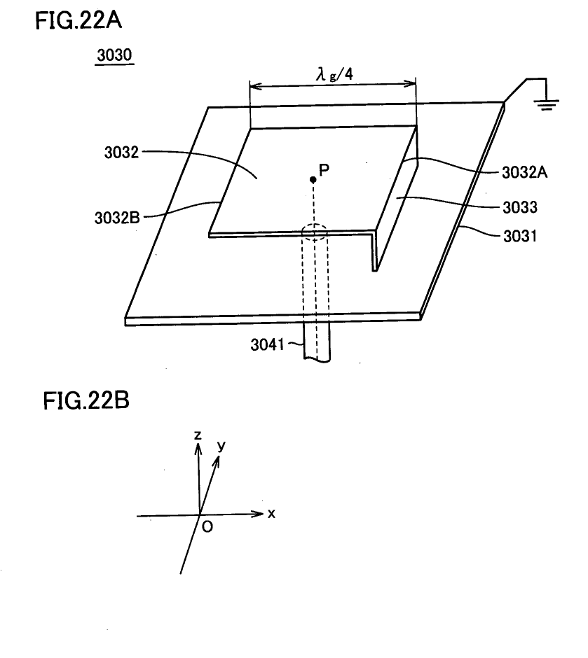

FIGS. 22A and 22B show a configuration of patch antenna 3030 shown in FIG. 21. Here, FIG. 22A is a perspective view of patch antenna 3030, while FIG. 22B shows coordinate systems.

Patch antenna 3030 is a monopole patch antenna, and as shown in FIG. 22A, it includes a ground plane 3031 formed of a grounded conductive plate, a conductive plate 3032 having a quadrangular two-dimensional shape for forming a resonator, and a conductive member 3033 connecting one end 3032A of conductive plate 3032 to ground plane 3031.

For ease of description, orthogonal coordinate systems are defined as follows. y axis is set to be parallel to one end 3032A of conductive plate 3032, x axis is set to be parallel to the other end that is adjacent to one end 3032A, and z axis is set in a direction leading from ground plane 3031 toward conductive plate 3032.

Conductive plate 3032 forming a resonator is quadrangular as described above. When the wavelength of the electromagnetic field in patch antenna 3030 is given as λg, then the length of conductive plate 3032 in x axis direction is set to approximately λg/4. Further, the length of conductive plate 3032 in y axis direction is preferably less than λg/2. Conductive plate 3032 is arranged facing to and parallel to ground plane 3031.

Conductive member 3033 connecting end 3032A of conductive plate 3032 to ground plane 3031 is a plate-like member provided perpendicular to ground plane 3031. The length of conductive member 3033 in y axis direction is equal to that of conductive plate 3032, and its length in z axis direction (i.e., height) is desirably 5-50 mm, approximately.

End 3032A of conductive plate 3032 is short-circuited to ground plane 3031 by conductive member 3033, and therefore the potential of end 3032A is fixed to 0 (zero). Hence, end 3032A is referred to as fixed end 3032A. On the other hand, end 3032B facing to fixed end 3032A is open, and hence it is referred to as open end 3032B.

Ground plane 3031, conductive plate 3032 and conductive member 3033 are formed of copper, aluminum or the like. Patch antenna 3030 with such a configuration is arranged such that conductive plate 3032 is located at the bottom facing to dielectric plate 12, as shown in FIG. 21.

As described above, coaxial line 3041 is used for feeding patch antenna 3030. As shown in FIG. 21, outer conductor 3042 of coaxial line 3041 is connected to ground plane 3031, while inner conductor (feed line) 3043 of coaxial line 3041 penetrates an opening of ground plane 3031 to be connected to conductive plate 3032 at feeding point P thereon. While feeding point P is provided at a point away from end 3032A of conductive plate 3032, it is desirable to be set in the vicinity of the center of conductive plate 3032, as shown in FIG. 22A, considering impedance matching and the like.

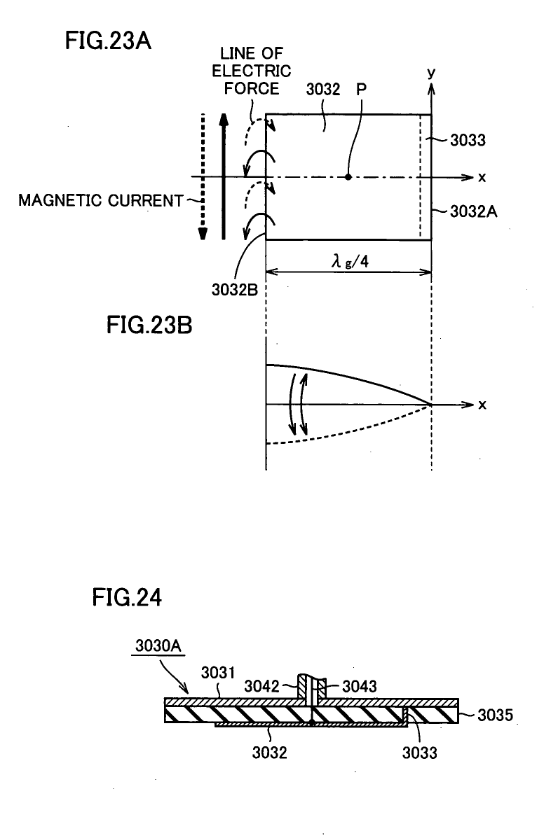

Next, the principle of radiation of electromagnetic wave by patch antenna 3030 is described. FIGS. 23A and 23B are illustrations therefor. FIG. 23A shows conductive plate 3032, while FIG. 23B shows the voltage distribution in x axis direction in conductive plate 3032.

The potential of fixed end 3032A of conductive plate 3032 is fixed to 0 (zero), and that the length of conductive plate 3032 in x axis direction is λg/4. Therefore, the current supplied from high frequency power supply 45 to conductive plate 3032 behaves as if the length of conductive plate 3032 in x axis direction is λg/2, oscillating in x axis direction to be a standing wave. Here, the voltage waveform repeats the change shown in FIG. 23B.

When the voltage at open end 3032B of conductive plate 3032 is positive, the line of electric force is directed from conductive plate 3032 toward ground plane 3031, and when the voltage at open end 3032B of conductive plate 3032 is negative, the line of electric force is directed from ground plane 3031 toward conductive plate 3032. The direction of the line of electric force is the same as the direction of the displacement current, and therefore a magnetic current is generated parallel to y axis as shown in FIG. 23A. With this magnetic current as a wave source, a high frequency wave is emitted from patch antenna 3030 in z axis direction.

Patch antenna 3030 has an excellent radiation efficiency of the electromagnetic field, since its magnetic current forming portion (i.e., the length of conductive plate 3032 in y axis direction) is longer as compared to conventional dipole antenna 530. From the view point of the radiation efficiency, the length of conductive plate 3032 in y axis direction should be longer. In order to decrease the effect of the magnetic current formed in the side face of patch antenna 3030 parallel to x axis, the length of conductive plate 3032 in y axis direction is preferably set to at least λg/8, approximately.

Next, an operation of the etching apparatus shown in FIG. 21 will be described.

The inside of processing container 11 is evacuated, for example, to a degree of vacuum of about 0.01-10 Pa, with substrate 21 placed on the upper surface of mounting table 22. While maintaining the degree of vacuum, Ar as the plasma gas is supplied from plasma gas supply nozzle 16, and CF₄ as the etching gas is supplied from processing gas supply nozzle 17, with the flow rate thereof being controlled.

When patch antenna 3030 is fed in the state where the plasma gas and etching gas is supplied into processing container 11, a high frequency wave is emitted from patch antenna 3030, as described above. Since patch antenna 3030 is shielded by ground plane 3031 in the upper direction, and by a shield member 18 in lateral directions, the high frequency wave passes through dielectric plate 12 and introduced into processing container 11. By forming the electric field in processing container 11 to cause electrolytic dissociation of Ar, plasma is generated in an upper space A above substrate 21 to be processed. The plasma diffuses in processing container 11, and the energy, anisotropy or the like thereof is controlled by the bias voltage applied to mounting table 22 to be used in the etching process.

Patch antenna 3030 used in this etching apparatus is a monopole antenna, and as compared to conventional dipole antenna 530, the size of the antenna can be reduced. When forming conductive plate 3032 to be a resonator by, for example, λg/4 angle, a processing container having a diameter L of approximately λg/4-λg/2 or a high frequency wave having a frequency of approximately c/(4 L)-c/(2 L), which cannot be used conventionally, can be utilized. Thus, the degree of freedom-in designing the etching apparatus that has been limited by the antenna size can be increased.

Though conductive plate 3032 of patch antenna 3030 shown in FIG. 22A is illustrated as quadrangular two-dimensionally, it is only required to have a substantially straight fixed end 3032A, and a length of approximately λg/4 in a direction perpendicular to fixed end 3032A (x axis direction). As used herein, substantially straight means a concept including not only a straight line but also a gentle curve. When fixed end 3032A is a gentle curve, open end 3032B opposing to fixed end 3032A may be a shape on which fixed end 3032A overlaps when translated. Additionally, conductive plate 3032 may be trapezoid or semi-circle two-dimensionally.

As shown in FIG. 24, a dielectric plate 3035 may be arranged between ground plane 3031 and conductive plate 3032 forming patch antenna 3030A. Thus, the wavelength λg of the electromagnetic field on conductive plate 3032 becomes shorter, which enables the patch antenna to be further smaller.

### Seventh Embodiment

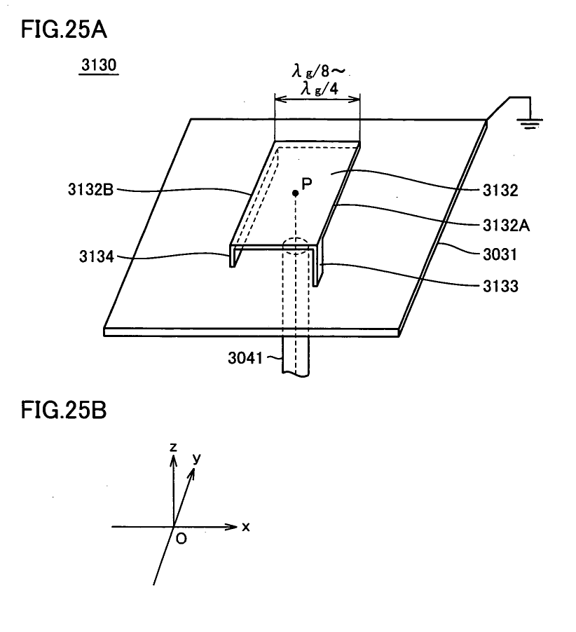

Next, a variation of patch antenna 3030 shown in FIG. 21 will be described. FIGS. 25A and 25B show a configuration of the variation. FIG. 25A is a perspective view, while FIG. 25B shows coordinate systems. In these figures, portions similar to those in FIGS. 22A and 22B are denoted by similar reference characters, and description thereof will appropriately be omitted.

Patch antenna 3130 shown in FIG. 25A has, in the configuration of patch antenna 3030 shown in FIG. 21, open end 3032B of conductive plate 3032 folded toward ground plane 3031, and conductive member 3031 partially cut out to be a narrow shape.

In the following, description will be made in detail. In patch antenna 3130, the length of conductive plate 3132 forming a resonator in x axis direction is at least approximately λg/8 and less than approximately λg/4.

Additionally, to conductive plate 3132, a conductive plate 3134 extending from open end 3132B toward ground plane 3031 is connected. The length of conductive plate 3134 in y axis direction is the same as that of conductive plate 3132, while the length thereof in z axis direction is shorter than that of conductive member 3133. Accordingly, the tip of conductive plate 3134 does not contact ground plane 3031. Further, conductive plate 3134 is made of the same material as conductive plate 3132 and the like, i.e., copper or aluminum.

Still further, conductive member 3133 connecting fixed end 3132A of conductive plate 3132 to ground plane is shorter in y axis direction than conductive plate 3132.

When patch antenna 3130 with such a configuration is fed, large capacitance is formed between conductive plate 3134 and ground plane 3031. Additionally, large inductance is formed in the narrow conductive member 3133. By designing the apparatus such that these capacitance and inductance compensate each other, the length of conductive plate 3132 in x axis direction can be shortened to approximately λg/8.

Though patch antenna 3130 is smaller than patch antenna 3030 shown in FIG. 21, it can emit a high frequency wave equivalent to those emitted by patch antenna 3030.

In patch antenna 3130 also, the patch antenna can be made further smaller by arranging dielectric plate 3035 as shown in FIG. 24 between ground plane 3031 and conductive plate 3132.

In the foregoing, while the example has been described where the plasma apparatus according to the third invention is applied to an etching apparatus, it is needless to say that it may be applied to other plasma apparatus such as a plasma CVD apparatus.

As described above, according to the plasma apparatus of the third invention, by forming the antenna supplying the high frequency electromagnetic field into the processing container by a monopole antenna to make the antenna smaller, the degree of freedom in designing the plasma apparatus can be increased, which has been limited by the size of the antenna.

Further, by using the patch antenna as the monopole antenna, the radiation efficiency of the high frequency wave can be improved.

In the following, referring to the figures, embodiments according to a fourth invention of the present invention will be described in detail. Here, an example will be described where a plasma apparatus according to the fourth invention is applied to an etching apparatus.

### Eighth Embodiment

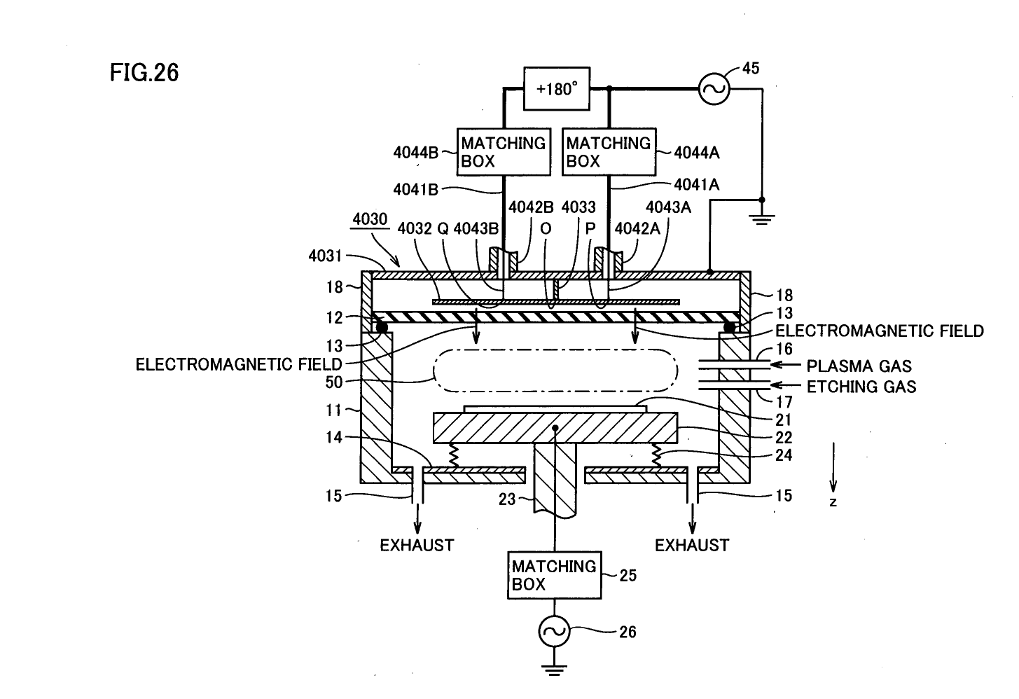

FIG. 26 shows a configuration of an etching apparatus according to an eighth embodiment of the present invention. FIG. 26 shows a cross section of a partial configuration thereof.

The etching apparatus shown in FIG. 26 has a cylindrical processing container 11 that is open at an upper portion. Processing container 11 is formed of a conductive member such as aluminum.

At the upper opening of processing container 11, a dielectric plate 12 formed of quartz glass or ceramic (such as Al₂O₃ or AlN) having a thickness of about 20-30 mm is arranged. At a joint portion between processing container 11 and dielectric plate 12, a sealing member 13 such as an O-ring is interposed, to ensure air-tightness inside processing container 11.

At the bottom of processing container 11, an insulating plate 14 formed of ceramic or the like is provided. Further, an exhaust port 15 is provided penetrating through insulating plate 14 and the bottom of processing container 11 and, by means of a vacuum pump (not shown) communicated with exhaust port 15, the inside of processing container 11 can be evacuated to a desired degree of vacuum.

At upper portion and lower portion of a sidewall of processing container 11, a plasma gas supply nozzle 16 is provided for introducing a plasma gas such as Ar and a processing gas supply nozzle 17 is provided for introducing an etching gas into processing container 11, respectively. Plasma gas supply nozzle 16 and processing gas supply nozzle 17 are formed by a quartz pipe or the like.

In processing container 11, a mounting table 22 is contained, on an mounting surface of which a substrate 21 to be etched (the object to be processed) is placed. Mounting table 22 is supported by an elevating shaft 23 passing, with a play, through the bottom of processing container 11 to be freely movable upward and downward. Further, mounting table 22 is connected to a biasing high frequency power supply 26 through a matching box 25. Output frequency of high frequency power supply 26 is a prescribed frequency within the range of several hundred kHz to ten and several MHz. In order to ensure air-tightness of processing container 11, a bellows 24 is provided to surround elevating shaft 23, between mounting table 22 and insulating plate 14.

Above dielectric plate 12, a patch antenna 4030 is arranged for supplying a high frequency electromagnetic field into processing container 11 through dielectric plate 12. Patch antenna 4030 is separated from processing container 11 by dielectric plate 12, and protected from the plasma generated in processing container 11. Further, a shield member 18 covers peripheries of dielectric plate 12 and patch antenna 4030 to prevent leakage of the high frequency electromagnetic field from patch antenna 4030 to the outside of the etching apparatus.

Patch antenna 4030 has ground plane 4031 formed of a grounded conductive plate, and a conductive plate 4032 forming a resonator (hereinafter referred to as a patch). Patch 4032 is arranged facing to ground plane 4031 with a prescribed distance, and the distance is maintained by a short pin 4033 connecting respective center. Ground plane 4031, patch 4032 and short pin 4033 are formed of copper, aluminum or the like. Patch antenna 4030 is arranged such that patch 4032 is located at the bottom facing to the mounting surface of mounting table 22 and dielectric plate 12.

Patch antenna 4030 is fed at two points. Two coaxial lines (first feeding lines) 4041A and 4041B are employed for the feeding. It is noted that coaxial line 4041B is longer in electrical length than coaxial line 4041A by 180°. As used herein, the electrical length is expressed by the phase difference when feeding electricity passes through coaxial lines 4041A, 4041B. In this case, it means that their feeding phases to patch antenna 4030 are different by 180°.

Coaxial lines 4041A and 4041B are connected to feeding high frequency power supply 45 through matching boxes 4044A and 4044B, respectively. Output frequency of high frequency power supply 45 is a prescribed frequency in a range of 100 MHz-8 GHz. Additionally, by interposing matching boxes 4044A and 4044B for improving impedance matching to coaxial lines 4041A and 4041B, respectively, the efficiency of power use can be improved.

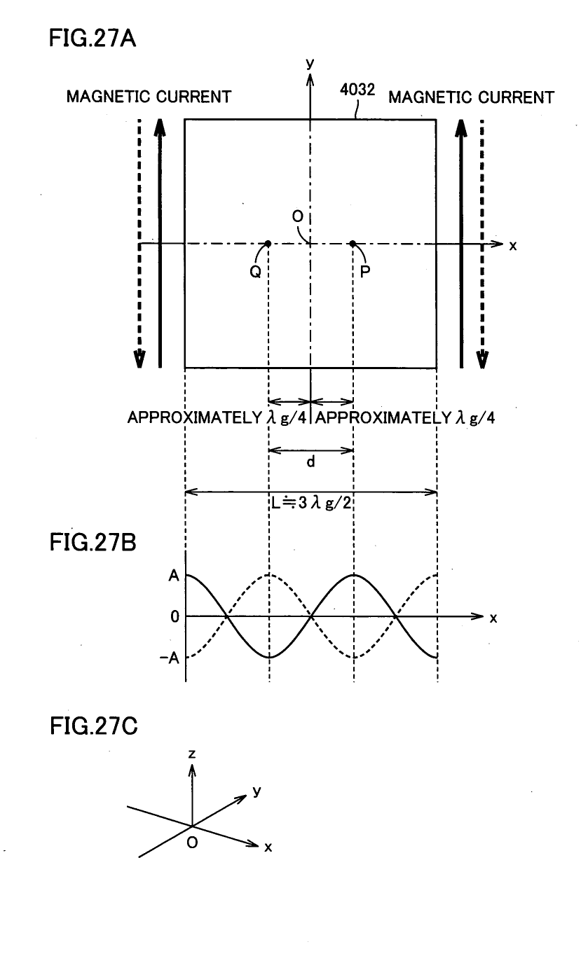

FIG. 27A is a plan view of patch 4032 in FIG. 26 viewed from the bottom. As shown in FIG. 27A, patch 4032 has a two-dimensional shape of a square in which the length L of one edge is approximately 3×λg/2. λg is a wavelength of the electromagnetic field between patch 4032 and ground plane 4031, of which value is determined by the permittivity between patch 4032 and ground plane 4031. Here, it is assumed that center O of patch 4032 is located at the origin of coordinate systems, while each edge of patch 4032 is parallel to x axis and y axis.

In this case, two feeding points P, Q of patch 4032 are provided at two points away from center O in the opposite directions by approximately λg/4 on x axis (first line). As shown in FIG. 26, as described above, feeding points P, Q are connected to inner conductors 4043A and 4043B of coaxial lines 4041A and 4041B, respectively, with coaxial line 4041B connected to feeding point Q being longer than coaxial line 4041A connected to feeding point B by 180°. It is noted that outer conductors 4042A and 4042B of coaxial lines 4041A and 4041B are connected to ground plane 4031.

Here, referring to FIGS. 27A-27C, the operating principle of patch antenna 4030 is described.

Two coaxial lines 4041A and 4041B are connected on x axis of patch 4032, with the length of patch 4032 in x axis direction being approximately 3×λg/2. Thus, the current supplied from two coaxial lines 4041A and 4041B resonates in x axis direction to be a standing wave. Here, by feeding at two feeding points P, Q, the mode of the standing wave is defined. The voltage waveform in x direction is as shown in FIG. 27B, with the opposing ends becoming antinodes and the number of waves being 3/2. Hence, the voltage variations at opposing ends show opposite phases. Accordingly, as shown in FIG. 27A, along opposing sides of patch 4032 in x axis direction, i.e., along two edges parallel to y axis, magnetic currents are generated in reverse directions as viewed from the center of patch 4032. Specifically, when one of the magnetic current is directed to the positive direction (or the negative direction) of y axis, the other magnetic current is also directed to the positive direction (or the negative direction) of y axis. Accordingly, in patch antenna 4030, only TM11 mode is excited, and TM01 mode is not excited. It is noted that high frequency wave is emitted having two magnetic currents as the wave source.

Next, an operation of the etching apparatus shown in FIG. 26 will be described.

The inside of processing container 11 is evacuated, for example, to a degree of vacuum of about 0.01-10 Pa, with substrate 21 placed on the mounting surface of mounting table 22. While maintaining the degree of vacuum, Ar as the plasma gas is supplied from plasma gas supply nozzle 16, and CF₄ as the etching gas is supplied from processing gas supply nozzle 17, with the flow rate thereof being controlled.

In the state where the plasma gas and the etching gas are supplied into processing container 11, patch antenna 4030 is fed at two feeding points P, Q in the same amplitude and with phases different by 180° from each other. Thus, patch antenna 4030 is selectively excited to TM11 mode. In TM11 mode, the directivity of high frequency electromagnetic field will be z axis direction perpendicular to the main surface (xy plane) of patch 4032, therefore the electromagnetic field is oriented directly toward substrate 21 to be etched.

The electric field causes electrolytic dissociation of Ar in processing container 11 to generate plasma in an upper space 50 above substrate 21 to be processed. The plasma diffuses in processing container 11, and the energy, anisotropy or the like thereof is controlled by the bias voltage applied to mounting table 22 to be used in the etching process.

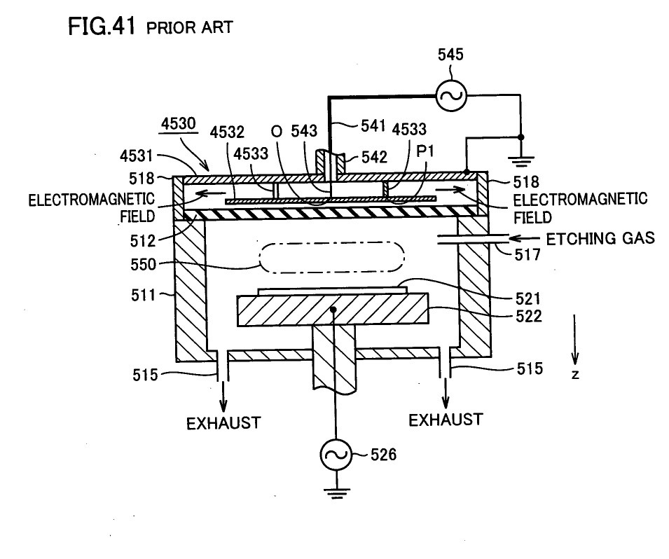

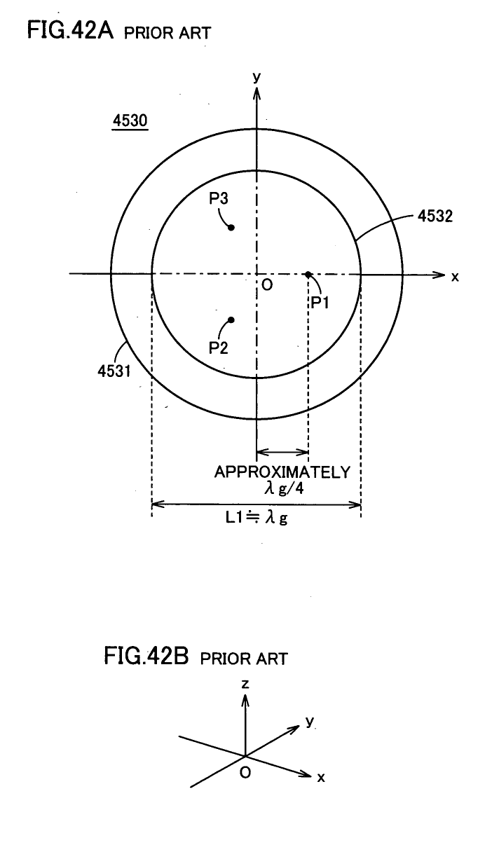

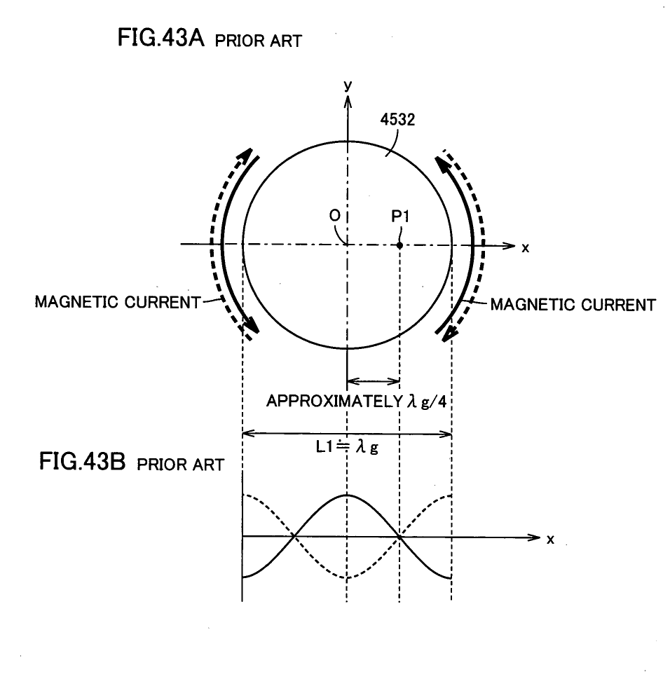

As described above, in this etching apparatus, the electromagnetic field is oriented directly toward substrate 21, therefore the power that is converted to thermal energy at shield member 18 or processing container 11 before contributing to plasma generation is decreased as compared to the conventional etching apparatus shown in FIG. 41. Thus, the power contributing to the plasma generation is increased. Accordingly, the efficiency of power at the plasma generation can be increased as compared to the conventional manner.

In FIG. 27A, it has been described that two feeding points P, Q are on x axis of patch 4032. This prevents the current from passing through y axis direction on patch 4032, which will suppress the radiation of a high frequency wave from two edges of patch 4032 parallel to x axis. Nevertheless, feeding points P, Q can be provided at points offset from x axis in a range in which the effect of the radiation can be permitted.

Additionally, though it has been described that two feeding points P, Q are provided away from center O on patch 4032 by the same distance, they may be provided at positions away from center O by different distances. However, since the potential will be 0 (zero) at the positions corresponding to nodes of a standing wave, it is not desirable to provide feeding points P, Q to such a position or in the vicinity thereof. Accordingly, it is desirable to provide feeding points P, Q at positions away from positions corresponding to the nodes of a standing wave by at least λg/16.

Further, since it is only required to define the mode of a standing wave generated in patch 4032 by two-point feeding, the distance d between two feeding points P, Q is not necessarily λg/2 and the feeding phase difference be 180°. Additionally, they are not necessarily correlated with each other. However, taking into account of the relationship between the nodes of the standing wave and feeding points P, Q as described above, the desirable minimum value of distance d between feeding points P, Q is approximately λg/8.

Still further, the length L of one edge of patch 4032 of patch antenna 4030 may be approximately (N+½)×λg (N is an integer at least 0).

Still further, two-dimensional shape of patch 4032 may be a quadrangle other than a square. In this case, when the length in x axis direction is L1≈(N+½)×λg, the length in y axis direction may be {(N′−1)+½}×λg<L2<(N′+½)×λg (N′ is an integer of 0≦N′≦N).

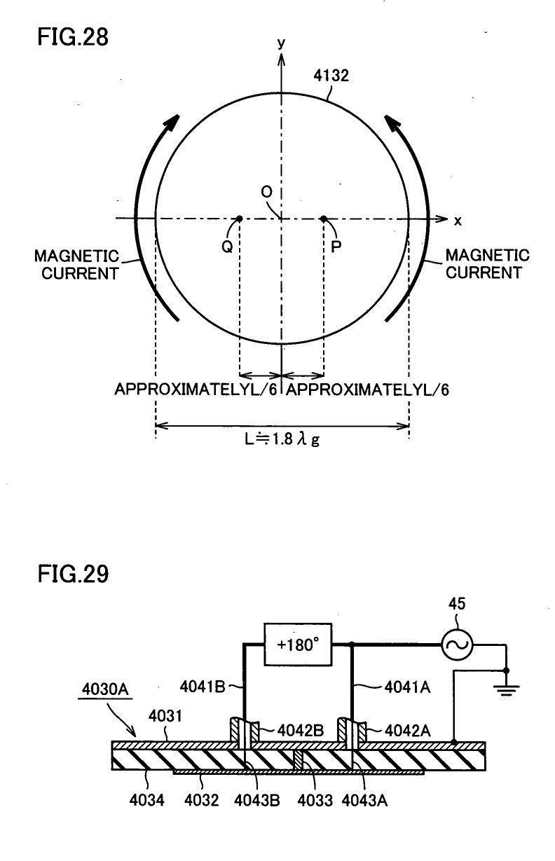

Still further, two-dimensional shape of the patch may be a circle as patch 4132 shown in FIG. 28. In this case, the diameter L of the circle may be approximately 1.17×(N+½)×λg. This dimension is a concept included in the approximate (N+½)×λg as described above. L≈1.8×λg shown in FIG. 28 is an example where N=1.

As shown in FIG. 29, a dielectric plate 4034 formed of ceramic or the like may be interposed between ground plane 4031 and patch 4032 forming patch antenna 4030. Thus, the patch antenna can be made smaller. In this case, short pin 4033 connecting patch 4032 and ground plane 4031 may not necessarily provided.

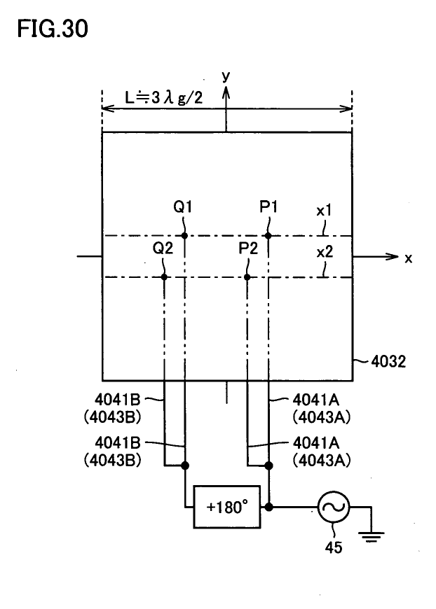

Still further, though FIG. 27A illustrates to provide two feeding points P, Q on x axis of patch 4032, feeding points may be provided two each (P1, Q1), (P2, Q2) on at least two lines (first lines) x1, x2 that are perpendicular to the outer periphery of patch 4032 as shown in FIG. 30. The matching box is omitted from FIG. 30.

### Ninth Embodiment

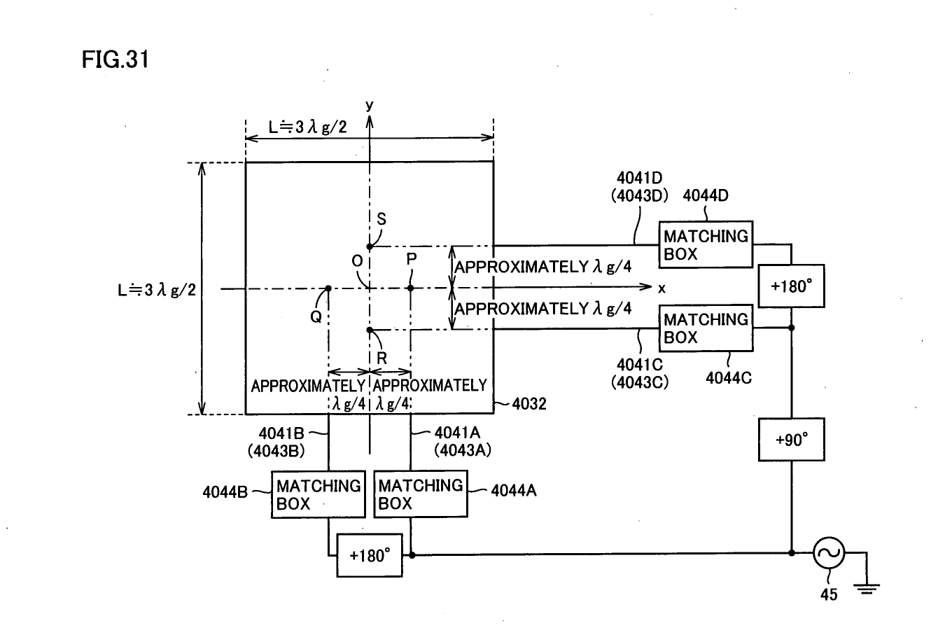

FIG. 31 shows a configuration for generating a circular polarization using patch antenna 4030 shown in FIG. 26. In this figure, portions similar to those of FIGS. 26, 27A-27C are denoted by the same reference characters, and description thereof will appropriately be omitted.

When generating a circular polarization, two additional feeding points R, S are provided on patch 4032 forming a resonator. These feeding points R, S are provided on y axis (second line) at two points away from center O by approximately λg/4 in opposite directions.

Inner conductors 4043C and 4043D of coaxial lines (second feeding lines) 4041C and 4041D are connected to feeding points R, S, with coaxial line 4041D connected to feeding point S being longer in electrical length than coaxial line 4041C connected to feeding point R by 180°. Additionally, coaxial lines 4041C and 4041D are longer in electrical length than coaxial lines 4041A and 4041B by 90°, respectively. Accordingly, the feeding point phase difference for feeding points R, S is 180°, and feeding points R, S are fed with phases lagging from feeding points P, Q by 90°, respectively. It is noted that matching boxes 4044C and 4044D are interposed in coaxial lines 4041C and 4041D, respectively.

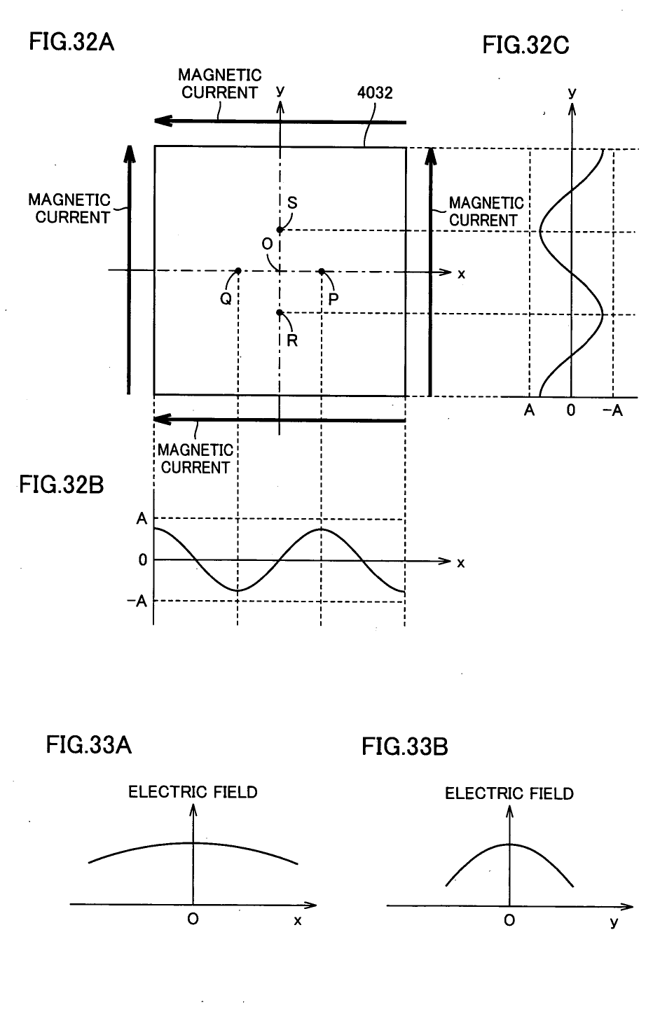

FIGS. 32A-32C illustrate the operating principle of patch antenna 4030 with four-point feeding as shown in FIG. 31, FIG. 32A shows a magnetic current generated around patch 4032, FIG. 31B shows the voltage waveform on x axis, and FIG. 31C shows the voltage waveform on y axis.

When patch 4032 is fed at two feeding points P, Q on x axis thereof with equal amplitude, then a high frequency wave is emitted having two magnetic currents parallel to y axis as the wave source, in the same principle described above with reference to FIGS. 27A-27C. This high frequency wave will be a linear polarization parallel to x axis. Similarly, when patch 4032 is fed at two feeding points R, S on y axis thereof with equal amplitude, then the high frequency wave is emitted having two magnetic currents parallel to x axis as the wave source. The high frequency wave will be a linear polarization parallel to y axis. At this time, feeding points Q, R are fed with phases lagging from feeding points P, Q by 90°, respectively, and therefore the phase of the linear polarization parallel to y axis lags by 90° from that of linear polarization parallel to x axis. These two linear polarizations have equal amplitude, perpendicular spatially, and out of phase by 90° to each other, and therefore they form a circular polarization. In this case, it will be a right-hand circular polarization in the vertical direction in FIG. 26 (positive direction in z axis).

When two-point feeding is employed as shown in FIGS. 26 and 27A, as the high frequency wave emitted by patch antenna 4030 will be a linear polarization parallel to x axis, the electric field distribution thereof will be as shown in FIGS. 33A and 33B. Specifically, though it is relatively uniform on xz plane as shown in FIG. 33A, it is biased on yz plane as shown in FIG. 33B.

Though bias is still present on the electric field distribution in the linear polarization parallel to x axis or y axis itself when four-point feeding is employed as shown in FIG. 31, by generating the circular polarization to rotate the electromagnetic field around the axis perpendicular to the mounting surface of mounting table 22, the plasma distribution generated by the electromagnetic field rotates as well. Thus, the etching process that is uniform when averaged by time can be attained.

It is noted that, when employing four-point feeding to generate the circular polarization, the two-dimensional shape of patch 4032 may be 90° rotationally symmetrical shape such as a square or a circle (a shape that overlaps when rotated by 90° around the central axis of patch 4032), or may be a shape having different lengths in two directions perpendicular to each other viewed from center O, such as a rectangle. In the case of the latter, the feeding phase difference of feeding points P, R and feeding points Q, S may not be set 90°, and it may be adjusted by the lengths of the two directions. In either case of the former and the latter, the two perpendicular lengths are required to be approximately (N+½)×λg, and approximately (M+½)×λg (N, M are integers at least 0).

Further, in the four-point feeding scheme shown in FIG. 31, it has been described that the right-hand circular polarization is emitted in the vertical direction of FIG. 26 (positive direction of z axis). To emit the left-hand circular polarization, coaxial lines 4041C and 4041D may be set to have electrical lengths longer than coaxial lines 4041A and 4041B by 90°, respectively.

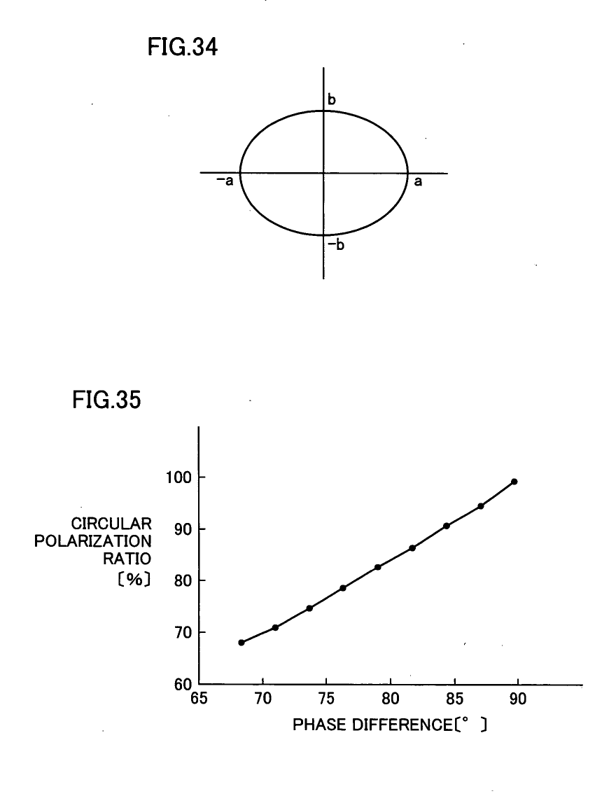

Still further, the high frequency wave emitted by the patch antenna 4030 is not necessarily a perfectly circular polarization. Given that the polarization ratio of a circular polarization of which length of long axis is 2a and that of short axis is 2b as shown in FIG. 34 is b/a (×100)%, generation of the circular polarization with a polarization ratio of at least 50%, preferably at least 70% results in the improvement of the plasma distribution.

Here, a method for adjusting the polarization ratio of a circular polarization is described briefly.

First, when two linear polarizations perpendicular to each other are out of phase to each other by 90° but with different amplitude values, given that the two linear polarizations are expressed as a sin (ωt+π/2), b sin (ωt), then the polarization ratio is simply determined by the amplitude value ratio b/a (×100)%. Therefore, in order to obtain the polarization ratio of at least 70%, the amplitude value ratio may only be set to at least 70%.

Additionally, when two linear polarizations perpendicular to each other are equal in amplitude value but with a phase difference other than 90°, given that the two linear polarizations are expressed as sin (ωt−θ), sin(ωt), then the phase difference dependency of the polarization ratio when the phase difference 0 takes on the value in the vicinity of 90° will be as shown in FIG. 35. Therefore, in order to obtain the polarization ratio of at least 70%, the phase difference 0 may only be adjusted to approximately 70°-110°.

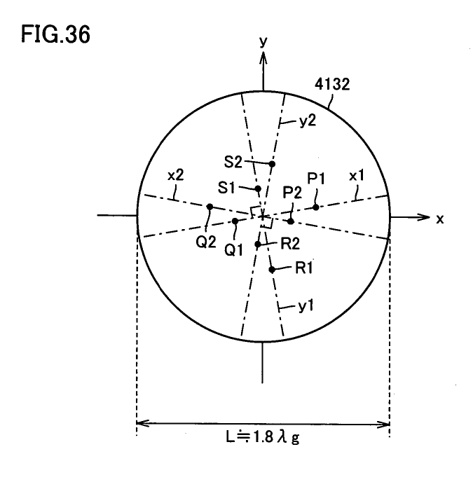

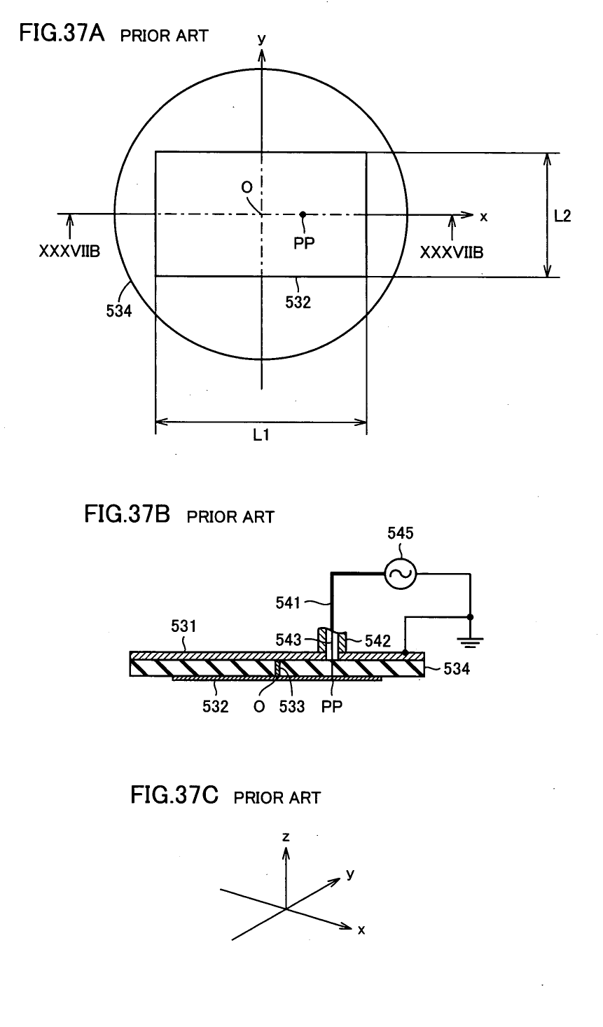

Further, when feeding-points are provided two each (P1, Q1), (P2, Q2) on two lines x1, x2 as shown in FIG. 30, feeding points are provided two each (R1, S1), (R2, S2) on two lines y1, y2 perpendicular to lines x1, x2, respectively, as in patch 4132 shown in FIG. 36. Then, feeding may be carried out such that the feeding phase differences between feeding points P1 and R1, feeding points Q1 and S1, feeding points P2 and R2, as well as feeding points Q2 and S2 are about the same.

In the foregoing, while the example has been described where the plasma apparatus according to the fourth invention is applied to an etching apparatus, it is needless to say that it may be applied to other plasma apparatus such as a plasma CVD apparatus.

As described above, in the plasma apparatus according to the fourth invention, the antenna is fed at two points to selectively excite the TM11 mode. Thus, the high frequency wave will be oriented directly to the object to be processed, which results in reduction of the power absorbed by the processing container or the like to increase the power that contributes in plasma generation. Hence, the efficiency of power in plasma generation can be improved.

Still further, by making the high frequency wave supplied from the antenna into the processing apparatus to the circular polarization, to rotate the electromagnetic field around the axis perpendicular to the mounting surface of mounting table, the plasma distribution generated by the electromagnetic field rotates as well. Thus, the uniformity of plasma distribution when averaged by time can be improved.

Although the present invention has been described and illustrated in detail, it is clearly understood that the same is by way of illustration and example only and is not to be taken by way of limitation, the spirit and scope of the present invention being limited only by the terms of the appended claims.

### INDUSTRIAL APPLICABILITY

The present invention may be applied to a plasma apparatus for processes such as oxide film formation, crystal growth of a semiconductor layer, etching and ashing, in manufacturing semiconductor devices. Accordingly, it can contribute to improving the semiconductor apparatus manufacturing technology.

1. A plasma apparatus, comprising: a mounting table arranged in an air-tight processing container for mounting an object to be processed; and an antenna arranged facing to said mounting table for supplying a high frequency wave into said processing container; wherein
said antenna has a conductive plate (1032) arranged facing to said mounting table to form a resonator, and a ground plane (1031) arranged facing to said conductive plate at a side opposite to said mounting table, and
said conductive plate is fed such that said high frequency wave becomes a circular polarization.

2. The plasma apparatus according to claim 1, wherein
said conductive plate is fed such that said high frequency wave becomes a circular polarization having a polarization ratio of at least 50%.

3. The plasma apparatus according to claim 1, further comprising
two feeding lines feeding said conductive plate.

4. The plasma apparatus according to claim 3, wherein
said two feeding lines feed such that two linear polarizations equal in amplitude, having different phases from each other by 90°, and spatially perpendicular to each other are emitted, respectively.

5. The plasma apparatus according to claim 4, wherein
said conductive plate has a two-dimensional shape of a 90° rotationally symmetrical shape, and
said two feeding lines are connected to said conductive plate at two points thereon away from a center of said conductive plate by substantially equal distances and in two directions perpendicular to each other as viewed from said center, for feeding in equal amplitude and with phases different from each other by 90°, respectively.

6. The plasma apparatus according to claim 1, wherein
said conductive plate has a two-dimensional shape with two different lengths in two directions perpendicular to each other as viewed from said center, said plasma apparatus further comprising
a feeding line connected to said conductive plate at one point thereon in a direction between said two directions, for feeding said conductive plate.

7. The plasma apparatus according to claim 6, wherein
said conductive plate has a two-dimensional shape of a circle of which peripheral region is cut out partially.

8. The plasma apparatus according to claim 6, wherein
said conductive plate has a two-dimensional shape of an ellipse or a quadrangle.

9. A plasma apparatus, comprising: a mounting table arranged in an air-tight processing container and having a mounting surface for mounting an object to be processed; and an antenna arranged facing to said mounting surface of said mounting table for supplying an electromagnetic field of a high frequency wave into said processing container; wherein
said antenna is formed of a plurality of monopole antennas, and configured such that said electromagnetic field forms a circular polarization.

10. The plasma apparatus according to claim 9, wherein
said electromagnetic field emitted by said antenna has a polarization ratio of at least 50%.

11. A plasma apparatus, comprising: a mounting table arranged in an air-tight processing container and having a mounting surface for mounting an object to be processed; and an antenna arranged facing to said mounting surface of said mounting table for supplying an electromagnetic field of a high frequency wave into said processing container; wherein
said antenna is formed of a plurality of monopole antennas, and configured such that said electromagnetic field forms a substantial TM01 mode.

12. The plasma apparatus according to claim 9, wherein
each of said plurality of monopole antennas is a patch antenna.

13. The plasma apparatus according to claim 12, wherein
said patch antenna includes
a conductive plate arranged facing to said mounting surface of said mounting table,
a ground plane arranged facing to said conductive plate at a side opposite to said mounting table as viewed from said conductive plate,
a conductive member connecting one end of said conductive plate to said ground plane, and
a feeding line connected to said conductive plate at a point away from said one end of said conductive plate, wherein
said one end of said conductive plate of said patch antenna is substantially straight, and a length in a direction perpendicular to said one end is at most approximately ¼ of a wavelength of the electromagnetic field in said patch antenna.

14. The plasma apparatus according to claim 13, wherein
said conductive plate of said patch antenna has other end opposing to said one end folded toward said ground plane, said one end being connected to said conductive member, and
said conductive member of said patch antenna has one length in a same direction as said one end of said conductive plate formed shorter than said one end.

15. The plasma apparatus according to claim 12, wherein
said patch antenna including
a conductive plate arranged facing to said mounting surface of said mounting table and having substantially straight one end and arc-shaped other end opposing to said one end, a length between said one end and said other end being at most (1.17±0.05)/4 of a wavelength of said electromagnetic field in said patch antenna,
a ground plane arranged facing to said conductive plate at a side opposite to said mounting table as viewed from said conductive plate,
a conductive member connecting said one end of said conductive plate to said ground plane, and
a feeding line connected to said conductive plate at a point away from said one end of said conductive plate.

16. The plasma apparatus according to 15, wherein
said conductive plate of said patch antenna has said other end folded toward said ground plane, and
said conductive member of said patch antenna has one length in a same direction as said one end of said conductive plate formed shorter than said one end.

17. A plasma apparatus, comprising:
a mounting table arranged in an air-tight processing container for mounting an object to be processed; and
an antenna arranged facing to said mounting table for supplying an electromagnetic field of a high frequency wave into said processing container; wherein
said antenna is a monopole antenna.

18. The plasma apparatus according to claim 17, wherein
said monopole antenna is a patch antenna.

19. The plasma apparatus according to claim 18, wherein
said patch antenna including
a conductive plate arranged facing to said mounting table and having substantially straight one end, a length of said conductive plate in a direction perpendicular to said one end being at most approximately ¼ of a wavelength of said electromagnetic field in said patch antenna,
a ground plane (3031) arranged facing to said conductive plate at a side opposite to said mounting table as viewed from said conductive plate,
a conductive member connecting said one end of said conductive plate to said ground plane, and
a feeding line connected to said conductive plate at a point away from said one end of said conductive plate.

20. The plasma apparatus according to claim 19, wherein
said conductive plate of said patch antenna has other end (3132B) facing to said one end connected to said conductive member folded toward said ground plane, and
said conductive member of said patch antenna has one length in a same direction as said one end of said conductive plate formed shorter than said one end.

21. The plasma apparatus according to claim 19, further comprising
a dielectric plate arranged between said conductive plate and said ground plane.

22. A plasma apparatus, comprising: a mounting table arranged in an air-tight processing container for mounting an object to be processed; and an antenna arranged facing to said mounting table for supplying a high frequency wave into said processing container; wherein
said antenna includes
a conductive plate arranged facing to said mounting table,
a ground plane arranged facing to said conductive plate at a side opposite to said mounting table as viewed from said conductive plate, and
a plurality of first feeding lines connected to said conductive plate, wherein
two each of said first feeding lines are connected away from each other to at least one first line on said conductive plate perpendicular to a periphery of said conductive plate, and
each of said first feeding lines has a length of approximately (N+½)×λg (N is an integer at least 0), where λg is a wavelength of an electromagnetic field between said conductive plate and said ground plane.

23. The plasma apparatus according to claim 22, wherein
said first line passes through a center of said conductive plate.

24. The plasma apparatus according to claim 22, wherein
said antenna further including includes
a plurality of second feeding lines connected to said conductive plate, two each of said second feeding lines being connected away from each other to at least one second line on said conductive plate perpendicular to corresponding said first line, wherein
each of said second lines has a length of approximately (M+½)×λg (M is an integer at least 0),
each of said second feeding lines feeds with a same degree of phase lag behind corresponding said first feeding lines, such that said high frequency wave becomes a circular polarization.

25. The plasma apparatus according to claim 24, wherein
each of said second feeding lines feeds such that said high frequency wave becomes a circular polarization having a polarization ratio of at least 50%.

26. The plasma apparatus according to claim 24, wherein
said first and second lines pass through a center of said conductive plate.

27. The plasma apparatus according to claim 22, wherein
a distance between said two first feeding lines connected to the same first line is λg/2.

28. The plasma apparatus according to claim 24, wherein
a distance between said two first feeding lines connected to the same first line, and a distance between said two second feeding lines connected to the same second line are each λg/2.
